(19) United States
(12) Patent Application Publication
(10) Pub. No.: US 2025/0228489 A1
(43) Pub. Date: Jul. 17, 2025

(54) SYSTEMS AND METHODS FOR AUTOMATED ELECTROCARDIOGRAM (ECG) INTERPRETATION OF WIDE COMPLEX RHYTHMS

(71) Applicant: Washington University, St. Louis, MO (US)

(72) Inventors: Adam May, St. Louis, MO (US); Anthony Kashou, Rochester, MN (US)

(21) Appl. No.: 18/980,157

(22) Filed: Dec. 13, 2024

Related U.S. Application Data
(60) Provisional application No. 63/609,406, filed on Dec. 13, 2023.

(51) Int. Cl. A61B 5/363; G16H 50/20

(52) U.S. Cl. CPC A61B 5/363; G16H 50/20

(57) ABSTRACT

A computer device for classifying a wide complex tachycardia (WCT) pattern of a subject is provided. The computer device is programmed to: a) receive WCT electrocardiogram (ECG) data indicative of a WCT pattern; b) transform the WCT ECG data into at least one engineering feature; c) execute at least one machine learning model to analyze the at least one engineering feature and to output a classification of the WCT pattern; d) based upon the classification of the WCT pattern, determine whether the WCT pattern is indicative of at least one of a ventricular tachycardia (VT) and a supraventricular wide complex tachycardia (SWCT); and e) select a treatment for the subject based upon the determination.

### CROSS REFERENCE TO RELATED APPLICATIONS

The present application claims the benefit of and priority to U.S. Provisional Patent Application No. 63/609,406, filed Dec. 13, 2023. The entire contents and disclosures of which is hereby incorporated by reference in its entirety.

### FIELD OF THE INVENTION

The present disclosure generally relates to computer-aided systems and methods for automatically interpreting wide complex rhythms in an electrocardiogram.

### BACKGROUND

Wide QRS complex tachycardia (WCT) is defined as a rapid rhythm with a ventricular rate greater than 100 beats per minute (bpm) and with a QRS duration of greater than 120 milliseconds (ms). The attributable causes of WCT include ventricular tachycardia (VT), supraventricular wide complex tachycardia (SWCT) due to pre-existing or functional aberrancy, SWCT arising from impulse propagation over atrioventricular accessory pathways (i.e., pre-excitation), tachycardias occurring with coexisting toxic metabolic QRS duration widening (e.g., hyperkalemia), and rapid ventricular pacing. It is critical that patient-facing clinicians accurately and promptly determine whether the WCT is due to VT or SWCT as there are important implications pertaining to immediate patient care decisions, subsequent clinical workup, and long-term management strategies.

The need for diagnostic tools to help clinicians accurately discriminate WCTs has been recognized for several decades. Numerous manual 12-lead electrocardiogram (ECG) interpretation algorithms and criteria have been developed to help clinicians distinguish VT from SWCT. While manual algorithms have generally demonstrated favorable diagnostic performance when applied by heart rhythm experts within highly regulated research settings, manual methods retain several important limitations when they are considered for broader clinical use. First, the diagnostic performance of manual methods is inextricably dependent on ECG interpreter competence and experience, which can be robust or lacking. Second, the unpracticed application of manual algorithms can be a problematic and time-consuming task for non-experts, one that is especially challenging for clinicians responsible for managing high-acuity and/or clinically deteriorating patients.

Although numerous WCT differentiation criteria and algorithms are readily available for clinical use, each universally possesses inherent and unavoidable limitations relating to their manual application. In genuine “real life” clinical circumstances, front-line clinicians must do the following to accurately differentiate WCTs by way of 12-lead ECG interpretation: (i) acquire and then visually inspect a properly recorded and well-timed 12-lead ECG that sufficiently ‘captures’ the WCT event, (ii) summon forth or precisely recall one or more of the available WCT differentiation criteria or algorithms, and (iii) carefully and diligently implement the chosen WCT differentiation method(s) without error, even while under duress.

Given the inherent challenges associated with manually operated WCT differentiation methods, automated solutions to distinguish VT and SWCT would be desirable.

This background section is intended to introduce the reader to various aspects of art that may be related to various aspects of the disclosure, which are described and/or claimed below. This discussion is believed to be helpful in providing the reader with background information to facilitate a better understanding of the various aspects of the present disclosure. Accordingly, these statements are to be read in this light, and not as admissions of prior art.

### BRIEF DESCRIPTION

Among the various aspects of the present disclosure is the provision of systems and methods for classifying a wide complex tachycardia (WCT) pattern, or other rhythms) of a subject.

The present teachings include a computer device for classifying a wide complex tachycardia (WCT) pattern of a subject. The computer device includes at least one processor in communication with at least one memory device. The at least one processor is programmed to receive WCT electrocardiogram (ECG) data indicative of a WCT pattern. The at least one processor is further programmed to transform the WCT ECG data into at least one engineering feature. The at least one processor is also programmed to execute at least one machine learning model to analyze the at least one engineering feature and to output a classification of the WCT pattern. Based upon the classification of the WCT pattern, the at least one processor is programmed to determine whether the WCT pattern is indicative of at least one of a ventricular tachycardia (VT) and a supraventricular wide complex tachycardia (SWCT). In addition, the at least one processor is programmed to select a treatment for the subject based upon the determination. The computer device may have additional, less, or alternate functionality, including that discussed elsewhere herein.

In another aspect, a computer device for classifying a wide complex tachycardia (WCT) pattern of a subject is provided. The computer device includes at least one processor in communication with at least one memory device. The at least one processor is programmed to receive WCT ECG data indicative of the WCT pattern and baseline ECG data. The at least one processor is also programmed to transform the WCT ECG data and baseline ECG data into at least one engineered feature. The at least one processor is further programmed to execute at least one machine learning model to analyze the at least one engineering feature and to output a classification of the WCT pattern. Based upon the classification of the WCT pattern, the at least one processor is programmed to determine whether the WCT pattern is indicative of at least one of a ventricular tachycardia (VT) and a supraventricular wide complex tachycardia (SWCT). In addition, the at least one processor is programmed to select a treatment for the subject based upon the determination. The computer device may have additional, less, or alternate functionality, including that discussed elsewhere herein.

In another aspect, a method for classifying a wide complex tachycardia (WCT) pattern of a subject is provided. The method is implemented by a computer device including at least one processor in communication with at least one memory device. The method includes receiving WCT ECG data indicative of the WCT pattern and baseline ECG data. The method also includes transforming the WCT ECG data and baseline ECG data into at least one engineered feature. The method further includes executing at least one machine learning model to analyze the at least one engineering feature and to output a classification of the WCT pattern. Based upon the classification of the WCT pattern, the method includes determining whether the WCT pattern is indicative of at least one of a ventricular tachycardia (VT) and a supraventricular wide complex tachycardia (SWCT). In addition, the method includes selecting a treatment for the subject based upon the determination. The method may have additional, less, or alternate functionality, including that discussed elsewhere herein.

In a further aspect, a computer-aided method of classifying a wide complex tachycardia (WCT) pattern of a subject is provided. In one aspect, the method can include receiving, using a computing device, WCT ECG data indicative of the WCT pattern. The method can further include transforming the WCT ECG data into at least one engineered feature. The method can further include classifying the WCT pattern based on the at least one engineered feature using a machine learning model. In some aspects, classifying the WCT pattern further comprises classifying the WCT pattern as indicative of at least one of a ventricular tachycardia (VT), a supraventricular wide complex tachycardia (SWCT), a probability of a VT; and a probability of an SWCT. In some aspects, the at least one engineered feature is selected from WCT QRS duration (ms), PMonoTVA (%), WCT Polarity Code (WCT-PC) for each lead of the ECG data, and any combination thereof. In some aspects, each WCT-PC for each lead of the ECG data is selected from positive, negative, or equiphasic. In some aspects, the machine learning model is selected from a logistic regression [LR] model, an artificial neural network [ANN], a Random Forests [RF] model, a support vector machine [SVM], and an ensemble learning [EL] model. The method may have additional, less, or alternate functionality, including that discussed elsewhere herein.

In further aspect, the method can include receiving, using a computing device, WCT ECG data indicative of the WCT pattern and baseline ECG data. The method can further include transforming the WCT ECG data and baseline ECG data into at least one engineered feature. The method can further include classifying the WCT pattern based on the at least one engineered feature using a machine learning model. In some aspects, classifying the WCT pattern further comprises classifying the WCT pattern as indicative of at least one of a ventricular tachycardia (VT), a supraventricular wide complex tachycardia (SWCT), a probability of a VT; and a probability of an SWCT. In some aspects, the at least one engineered feature is selected from WCT QRS duration (ms), PMonoTVA (%), QRS-PS for each lead of the ECG data, and any combination thereof. In some aspects, each QRS-PS for each lead of the ECG data is selected from equiphasic (=)→equiphasic (=), positive (+)→positive (+), negative (−)→negative (−), positive (+)→negative (−), positive (+)→equiphasic (=), negative (−)→positive (+), negative (−)→equiphasic (=), equiphasic (=)→positive (+), or equiphasic (=)→negative (−). In some aspects, the machine learning model is selected from a logistic regression [LR] model, an artificial neural network [ANN], a Random Forests [RF] model, a support vector machine [SVM], and an ensemble learning [EL] model. The method may have additional, less, or alternate functionality, including that discussed elsewhere herein.

In further aspect, the method can include receiving, using a computing device, WCT ECG data indicative of the WCT pattern and baseline ECG data. The method can further include transforming the WCT ECG data and baseline ECG data into at least one engineered feature. The method can further include classifying the WCT pattern based on the at least one engineered feature using a machine learning model. In some aspects, classifying the WCT pattern further comprises classifying the WCT pattern as indicative of at least one of a ventricular tachycardia (VT), a supraventricular wide complex tachycardia (SWCT), a probability of a VT; and a probability of an SWCT. In some aspects, the at least one engineered feature is selected from WCT QRS duration (ms), PMonoTVA (%), QRS-PS for each lead of the ECG data, and any combination thereof. In some aspects, each WCT-PC for each lead of the ECG data is selected from positive, negative, or equiphasic. In some aspects, each QRS-PS for each lead of the ECG data is selected from equiphasic (=)→equiphasic (=), positive (+)→positive (+), negative (−)→negative (−), positive (+)→negative (−), positive (+)→equiphasic (=), negative (−)→positive (+), negative (−)→equiphasic (=), equiphasic (=)→positive (+), or equiphasic (=)→negative (−). In some aspects, the machine learning model is selected from a logistic regression [LR] model, an artificial neural network [ANN], a Random Forests [RF] model, a support vector machine [SVM], and an ensemble learning [EL] model. The method may have additional, less, or alternate functionality, including that discussed elsewhere herein.

Advantages will become more apparent to those skilled in the art from the following description of the embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the systems and methods disclosed. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals. There are shown in the drawings arrangements presently discussed, it being understood, however, that the present embodiments are not limited to the precise arrangements.

FIG. 1 illustrates a flow diagram of a process 100 for wide QRS complex tachycardia (WCT) Differentiation in accordance with at least one embodiment.

FIG. 2 illustrates a simplified block diagram of an exemplary computer system for implementing the process shown in FIG. 1.

FIG. 3 illustrates a component configuration of a computing device, which includes database 310 along with other related computing components.

FIG. 4 illustrates an exemplary configuration of a client computer device shown in FIG. 2, in accordance with one embodiment of the present disclosure.

FIG. 5 illustrates an exemplary configuration of a server shown in FIG. 2, in accordance with one embodiment of the present disclosure.

FIG. 6 illustrates an exemplary table describing the characteristics of the training cohort from Institution 1.

FIG. 7 illustrates an exemplary table describing the characteristics of the training cohort from Institution 2.

FIG. 8 illustrates an exemplary table summarizing the diagnostic performance of various ML model subtypes in Part 1 (using 14 features) on gold standard and non-gold standard cohorts of the testing cohort (from Institution #2).

FIG. 9 illustrates an exemplary table summarizing the diagnostic performance of various ML model subtypes in Part 2 (using 14 features) on gold standard and non-gold standard cohorts of the testing cohort (from Institution #2).

FIG. 10 illustrates an exemplary table summarizing the diagnostic performance of various ML model subtypes in Part 3 (using 26 features) on gold standard and non-gold standard cohorts of the testing cohort (from Institution #2).

FIGS. 11A-11C illustrates an exemplary schematic of the three-part study design described in the current disclosure.

FIG. 12 illustrates an exemplary schematic describing how the WCT Polarity Codes were determined by way of summating QRS complex waveform amplitudes (μV) above (r/R and r′/R′) and below (q/QS, s/S, and s′/S′) the isoelectric baseline.

FIG. 13 illustrates an exemplary schematic showing examples of QRS polarity shift possibilities during the transition of a baseline ECG into a WCT ECG, and vice versa.

FIG. 14 illustrates an exemplary table showing categorical subtypes for QRS-Polarity Shift.

FIG. 15 illustrates an exemplary set of schematics showing examples of QRS Polarity Shift (QRS-PS) among examples of VT and SWCT.

FIGS. 16A and 16B illustrates an exemplary pair of forest plots demonstrating the discriminatory capacity of positive and negative WCT-PCs among individual ECG leads.

FIGS. 17A and 17B illustrates an exemplary pair of forest plots demonstrating the discriminatory capacity QRS-PS among individual ECG leads.

FIG. 18 illustrates an exemplary schematic of the proposed process for the WCT differentiation model application as determined by the presence or absence of a baseline ECG.

FIG. 19 illustrates an exemplary table summarizing the ability of the models of the current disclosure to differentiate between SWCT and VT.

FIGS. 20A and 20B illustrates an exemplary table showing frequency of QRS-PS among 12-lead ECG leads categorized according to 9 specific subtypes.

FIGS. 21A-21D illustrates an exemplary table showing the frequency of QRS-PS among the leads of the 12-lead ECG categorized into three general subtypes: [i] no polarity shift, [ii] polarity shift, and [iii] partial polarity shift.

FIG. 22 illustrates an exemplary table summarizing the diagnostic performance of various ML model subtypes for Study Part 1.

FIG. 23 illustrates an exemplary table summarizing the tuning parameters for ML model subtypes in Study Part 1 of the present disclosure.

FIG. 24 illustrates an exemplary table summarizing the diagnostic performance of various ML model subtypes in Study Part 2 of the present disclosure.

FIG. 25 illustrates an exemplary table summarizing the tuning parameters for ML model subtypes in Study Part 2.

FIG. 26 illustrates an exemplary table summarizing the diagnostic performance of ML Model Training in Study Part 3

FIG. 27 illustrates an exemplary table summarizing the tuning parameters for ML model subtypes in Study Part 3.

FIG. 28 illustrates an exemplary flow diagram depicting WCT ECG cohort selection (Institution #2).

FIG. 29 illustrates an exemplary is a schematic of measurements of QRS complex waveforms showing a schematic representation of a QRS complex and its measurable components provided by computerized ECG interpretation software.

FIG. 30 illustrates an exemplary schematic flow chart of the percent monophasic time-voltage area calculation.

FIG. 31 illustrates an exemplary table of importance scores for RF Models in Part 1.

FIG. 32 illustrates an exemplary table of importance scores for RF Models in Part 2.

FIG. 33 illustrates an exemplary table of importance scores for RF Models in Part 3.

Corresponding reference characters indicate corresponding parts throughout the drawings.

### DETAILED DESCRIPTION

The systems and methods of the present disclosure are based on the discovery of machine learning methods to automatically differentiate wide complex tachycardias (WCTs) into ventricular tachycardia (VT) and supraventricular wide complex tachycardia (SWCT). Given the inherent challenges associated with manually operated WCT differentiation methods, automated solutions to distinguish VT and SWCT are disclosed herein, including the WCT Formula, the VT Prediction Model, and the WCT Formula II. The fundamental characteristic of each proposed method is the integration of mathematically formulated parameters (e.g., frontal percent amplitude change (PAC) or horizontal percent time-voltage area change (PTVAC)) each directly derived from computerized ECG measurement data, into an automatic binary classification model. These automated WCT differentiation models provide an effective means to differentiate WCTs accurately once they are integrated into existing computerized ECG interpretation software systems. The systems and methods described herein enable automatic and vastly superior WCT differentiation accuracy compared with conventional manual methods.

In some aspects, the present disclosure includes a method of transforming readily available computerized ECG data into engineered features or parameters, which are based upon underpinning electrophysiologic principles. In other aspects, the engineered features or parameters can be leveraged by machine learning algorithms to accomplish automatic and accurate VT and SWCT classification using computerized ECG interpretation software.

In some aspects, the present disclosure details a process of converting readily available ECG measurement data (i.e., QRS waveform measurements) into engineered features that can be used by automated classification models (logistic regression, artificial neural networks, Random Forests, support vector machines, etc.) intended to classify VT or SWCT (i.e., WCT differentiation).

In accordance with another aspect, the methods use engineered features (e.g., Percent monophasic TVA) that can be combined with other previously described features (e.g., Percent amplitude change) to enhance accurate and automatic WCT differentiation.

In other aspects, the methods can be used to offer an estimation of the likelihood (i.e., probability or odds) of VT or SWCT to medical providers. Such an estimation would serve as cognitively meaningful clinical data that could be integrated with the provider's use of traditional (i.e., manual) methods to differentiate WCTs (e.g., Brugada algorithm).

In yet other aspects, the methods can be based upon and leverage a cognizable underlying electrophysiology principle (i.e., SWCT and VT have fundamental differences in the extent and efficiency to which they utilize the heart's native conduction system).

In other aspects, accurate and automatic WCT differentiation may be accomplished through the use of engineered features derived from computerized data of (i) WCT ECG alone and (ii) paired WCT and baseline ECGs. Once automated, the disclosed WCT differentiation models, which incorporate predictive engineered features derived from computerized ECG data, are suitable for integration into ECG interpretation software platforms to help clinicians distinguish VT and SWCT accurately and quickly select appropriate treatment.

In various aspects, systems and methods for wide QRS complex tachycardia (WCT) differentiation into ventricular tachycardia (VT) and supraventricular wide QRS tachycardia (SWCT) are suitable for use with patients with and without a baseline ECG are disclosed herein. The disclosed systems and methods make use of at least several existing engineered features disclosed herein as well as two additional engineered features disclosed herein: percent monophasic time-voltage area (PMonoTVA) and percent monophasic amplitude (PMonoAmp). In some aspects, the engineered features are derived from computerized ECG measurements as described herein. In various aspects, the disclosed engineered features provide for the differentiation of WCTs based on the WCT ECG alone without the need for a comparison baseline ECG. In various additional aspects, the engineered features described herein are suitable for incorporation into automated WCT differentiation models applicable to patients who present with or without corresponding baseline ECGs.

In various aspects, the disclosed systems and methods provide an estimated likelihood (i.e., probability or odds) of VT or SWCT to medical providers based on the automated analysis of WCT ECG. This estimated likelihood serves as cognitively meaningful clinical data that could supplement or replace information obtained by a provider using traditional (i.e., manual) methods to differentiate WCTs including, but not limited to, the Brugada algorithm.

In various aspects, the disclosed WCT differentiation systems and methods overcome at least some of the shortcomings of existing manual and automated WCT differentiation systems. In some aspects, the disclosed systems and methods enhance the diagnostic capability of automated electrocardiogram (ECG) interpretation, a critical component that supports clinical decision-making by medical providers. In other aspects, the disclosed systems and methods provide prompt and accurate wide complex tachycardia (WCT) differentiation into ventricular tachycardia (VT) and supraventricular wide complex tachycardia (SWCT), which is an undeniably common, vital, and challenging clinical task. In other additional aspects, the disclosed systems and methods automatically differentiate wide complex tachycardias (WCTs) into ventricular tachycardia (VT) and supraventricular wide complex tachycardia (SWCT), thereby reducing the variability and potential error introduced by manual methods and freeing up the practitioners to attend to the treatment of their patients.

In various aspects, the disclosed systems and methods transform readily available computerized ECG data into engineered features as described in additional detail herein. Without being limited to any particular theory, these engineered features capture and quantify cognizable underlying electrophysiology principles, namely that SWCT and VT are characterized by fundamental differences in the extent and efficiency to which they utilize the heart's native conduction system. In various aspects, these engineered features are suitable for analysis using machine learning algorithms or other automated classification models to accomplish automatic and accurate VT and SWCT classification using computerized ECG interpretation software.

In various aspects, various suitable machine learning algorithms or other automated classification models may be used by the disclosed systems and methods to differentiate WCTs without limitation. Non-limiting examples of suitable machine learning algorithms or other automated classification models include logistic regression, artificial neural networks, random forest models, support vector machines, and any other suitable ML or other automated classification algorithms.

In another aspect, automated differentiation of wide QRS complex tachycardia using QRS complex polarity is described, of which further details can be found in Example 1 of the present disclosure.

Although the disclosed methods are described herein in terms of the differentiation of wide QRS complex tachycardias, it is to be understood that the methods may be modified to enable the differentiation of a variety of isolated wide complex beats, in addition to the wide complex tachycardias that are typically characterized by an extended series of wide complex beats. Such isolated wide complex beats could be differentiated into premature ventricular contractions (PVC) and premature supraventricular contractions.

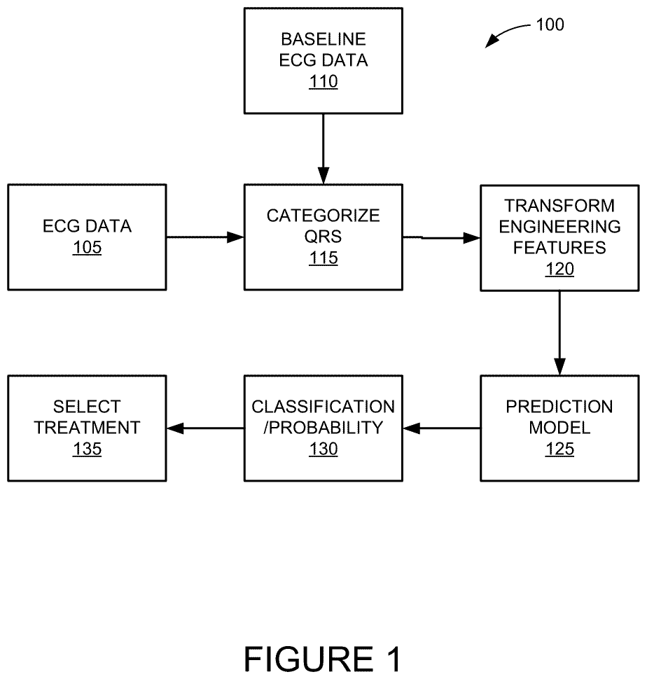

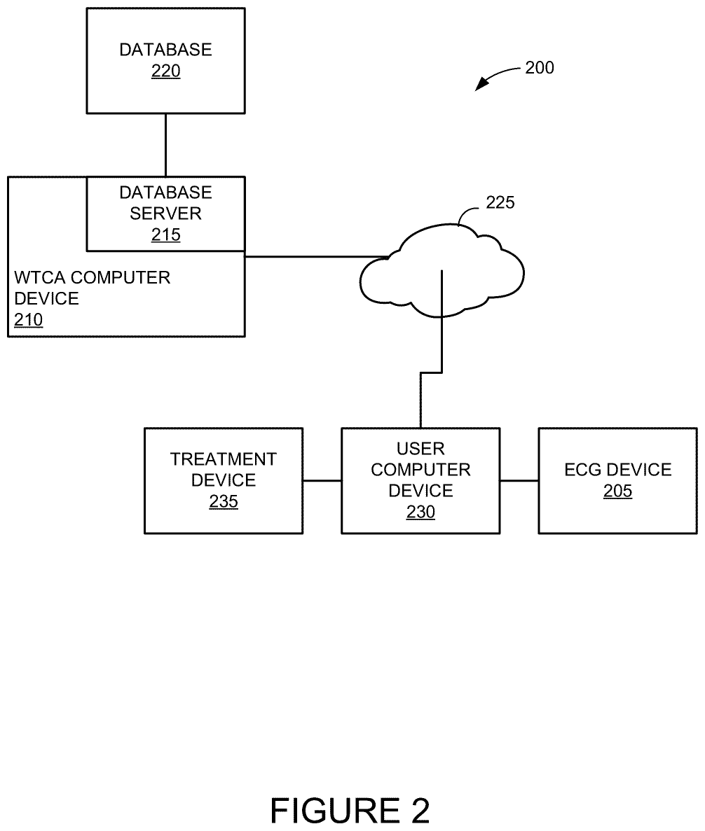

FIG. 1 illustrates a flow diagram of a process 100 for wide QRS complex tachycardia (WCT) Differentiation in accordance with at least one embodiment. In the exemplary embodiment, the steps of process 100 are performed by a wide QRS complex tachycardia analysis (WCTA) computer device 210, as shown in FIG. 2.

In at least one embodiment, the process 100 describes identifying ventricular tachycardia (VT) or supraventricular wide QRS tachycardia (SWCT) in a patient with wide QRS complex tachycardia (WCT) based on the patient's electrocardiogram (ECG) readings 105. In some aspects, the ECG readings 105 are WCT ECG readings obtained from the patient during wide QRS complex tachycardia (WCT). In other aspects, the ECG readings 105 further include baseline ECG readings 110 obtained from the patient outside of the WCT event in addition to the WCT ECG readings 105.

In these embodiment, the disclosed process 100 transforms WCT ECG readings 105, or alternatively WCT ECG readings 105 in combination with baseline ECG readings 110 into a classification of the WCT as VT or SWCT and/or a probability that the WCT is a VT or SWCT. As described herein, the information obtained using the disclosed method may be used by a practitioner to inform the selection of an appropriate treatment strategy.

Process 100 can include receiving 12-lead WCT ECG data 105 and automatically categorizing 115 the QRS complex as either monophasic or multiphasic for the ECG data 105 from each of the twelve leads. The QRS data is then transformed 120 into one or more of the engineered features, and one or more of these engineered features are incorporated into one of the predictive models 125 described herein. The predictive models can be trained with supervised learning, where the predicted WCT rhythm classification determined is compared to a previously determined classification to assess the accuracy of the predictive model 125. In some aspects, the process 100 is performed iteratively for a population of patients, and the predictive model 125 may be refined iteratively by evaluating predictive models 125 produced using different combinations of engineered features determined.

The process 100 also includes the WCTA computer device 210 receiving baseline ECG data 110 and/or WCT ECG data 105. The WCTA computer device 210 categorizes 115 the QRS data. In some embodiments, the WCTA computer device 210 transforms 120 the baseline ECG data 110 and/or WCT ECG data 105 into engineered features using predetermined relationships. In other embodiments, the ECG device 205 may further provide for the transformation of the ECG measurements into at least one engineered parameter using automated data analysis software.

Then the WCTA computer device 210 uses the predictive models 125 to transform the engineered features into a classification or estimated probability 130 of VT or SWCT. In some embodiments, the process 100 further includes the WCTA computer device 210 selecting 135 a treatment based on the classification or probability 130 of VT or WCT obtained. In some embodiments, the process 100 accomplishes the WCT differentiation based solely on the WCT ECG data 105 alone. In other embodiments, the process 100 accomplishes the WCT differentiation based on the WCT ECG data 105 as well as baseline ECG data 110.

In some aspects, the WCT ECG 105 and/or baseline ECG data 110 comprise the voltage readings obtained by the ECG system or device 205 (shown in FIG. 2) as described herein. In other aspects, the WCT ECG 105 and/or baseline ECG data 110 further comprise at least one summary parameter including, but not limited to, the durations and/or amplitudes characterizing a QRS complex waveform as described herein. In other aspects, where baseline ECG data 110 is available, the WCT ECG data 105 and baseline ECG data 110 further comprise at least one comparison parameter including, but not limited to, differences between corresponding summary parameters obtained from the WCT ECG data 105 and the baseline ECG data 110, respectively.

In various aspects, the ECG data 105 is measured using any suitable ECG measurement system or device 205 without limitation. Non-limiting examples of suitable ECG devices 205 include, but are not limited to, a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automatic implantable cardioverter defibrillator (AICD), an automated external defibrillator (AED), and any other suitable ECG measurement system or device 205.

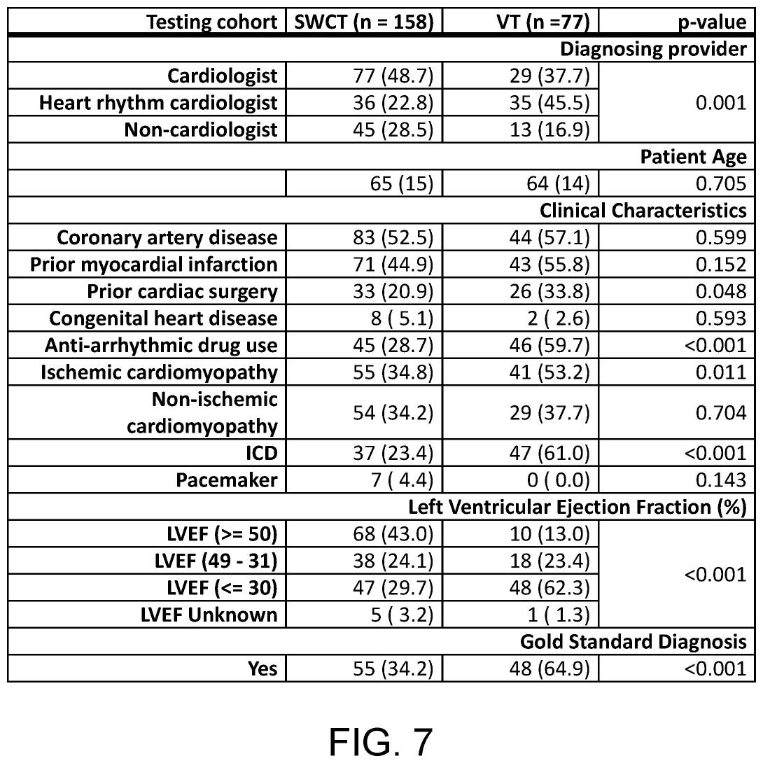

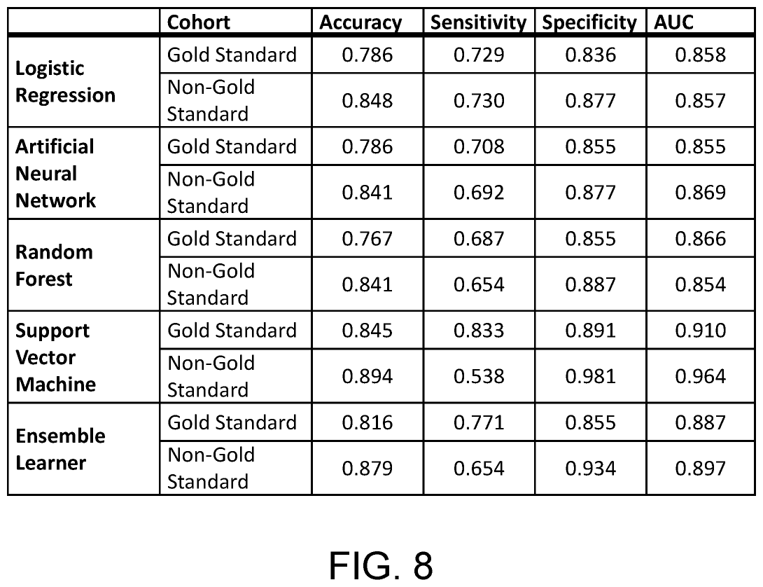

In one aspect, the ECG data 105 is measured using a 12-lead ECG device 205. The 12 ECG leads can be denoted according to their positioning on the patient as V1, V2, V3, V4, V5, V6, I, aVL, II, aVF, III, and aVR. Each of the 12 leads records a unique QRS complex representative of the heart's ventricular depolarization, and the ECG recordings from the ECG leads or various combinations thereof may be analyzed using the disclosed method as described herein. As described in further detail herein, the voltage amplitude measurements from specific leads including, but not limited to, frontal ECG plane leads V1, V4, V6 and horizontal ECG plane leads aVL, aVF, aVR are used in the formulas to generate engineered features including, but not limited to, frontal PACs and horizontal PACs. In other additional aspects, frontal PTVAC (shown in FIG. 7 and horizontal PTVAC shown in FIG. 8) are similarly generated.

The QRS complex waveform can be a combination of three graphical deflections: the Q wave having a downward deflection immediately following the P wave; the R wave having an upward deflection immediately following the Q wave; and the S wave having a downward deflection following the R wave. The Q wave, R wave, and S wave occur in rapid succession in a QRS complex waveform that occurs over a time interval denoted as QRS duration. A T wave follows the S wave. Each wave has amplitude denoted as PA, QA, RA, SA, and TA. In addition, the QT interval is the time interval extending from the onset of the QRS complex waveform to the end of the T wave. The QRS complex is divided into positive amplitudes and negative amplitudes. The positive amplitudes are the vertical QRS complex deflections above the isoelectric baseline, namely the amplitudes of the r/R wave and r′/R′ wave. The negative amplitudes are the vertical QRS complex deflections below the isoelectric baseline, namely the amplitudes of the q wave or QS wave, the s/S wave, and the s′/S′ wave.

In other aspects, the QRS complex can be divided into positive time-voltage areas (TVA) and negative TVAs, which may be marked as blue and yellow regions, respectively. The positive TVAs are the TVAs of the vertical QRS complex deflections above the isoelectric baseline, namely the TVAs of the r/R wave (and r′/R′, not shown). The negative TVAs are the TVAs of the vertical QRS complex deflections below the isoelectric baseline, namely the TVAs of q (or QS, not shown) and s/S (and s′/S′, not shown) waves. Computerized ECG interpretation software automatically measures QRS complex waveform attributes, namely q or QS, r/R, s/S, r′/R′, s′/S′ durations (ms), amplitudes (μV), and time-voltage areas (μV·ms).

In some aspects, automated data analysis software provides QRS complex waveform attributes, including, but not limited to, q or QS, r/R, s/S, r′/R′, s′/S′ durations (ms), amplitudes (μV), and time-voltage areas (μV·ms). By way of non-limiting example, automated data analysis software may provide the amplitudes (_A), durations (_D), and voltage areas (_VA) for the various waves including, but not limited to, values denoted as PA, PPA, QA, QD, RA, RD, SA, SD, RPA, RPD, SPA, and SPD. It is to be noted that the measurements described herein are not routinely shown on the ECG paper recordings, but are available within the ECG interpretation software databanks.

In some aspects, if baseline ECG data 110 is available for a patient, the baseline ECG data 110 may be compared to the WCT WCG data 105 to derive additional comparison parameters indicative of variations in the heart's ventricular depolarization relative to a normal ventricular depolarization. In various aspects, the comparison parameters include any changes in any of the QRS waveform parameters relative to corresponding baseline QRS waveform parameters as described herein without limitation. In some aspects, these comparison parameters are produced by the WCTA computer device 210. By way of non-limiting example, WCTA computer device 210 may produce a variety of comparison parameters including, but not limited to differences in QRS duration (ms), changes in frontal plane R wave axis) (°, and changes in frontal plane T wave axis) (° between the WCT 105 and baseline ECG data 110.

Step 120 of process 100 describes transforming 120 the baseline ECG data 110 and/or WCT ECG data 105 into engineered features. In various aspects, the engineered features comprise various combinations of subsets of the waveform attributes described herein. In various aspects, the subsets of waveform attributes comprise corresponding waveform attributes derived from the voltage measurements obtained by at least two different leads of the baseline ECG data 110 and/or WCT ECG data 105.

In various aspects, the engineered features are provided in the form of parameter values produced according to a predetermined relationship. Any suitable predetermined relationship may be used to produce the parameter values without limitation including, but not limited to, a data analysis method, an algorithm, or an equation. Non-limiting examples of engineered features suitable for use in the methods disclosed herein are described in PCT International Publication No. WO/2020/01471 and PCT/US2022/048618, the contents of which are incorporated by reference herein in their entirety.

In various aspects, the engineered features include PMonoTVA which quantifies the proportion of the time-voltage areas (TVA) of the QRS waveforms that include monophasic QRS complexes within the WCT ECG data 105. In some aspects, the TVA of the QRS waveforms that include monophasic QRS complexes may be calculated. In other aspects, the engineered features further include PMonoAmp which quantifies the proportion of amplitudes of the QRS waveforms that include monophasic QRS complexes within the WCT ECG data 105.

Percent monophasic time-voltage area (PMonoTVA), as used herein, refers to the percentage of QRS TVA contained by monophasic QRS complexes of the 12-lead ECG. In various aspects, PMonoTVA is calculated as the percentage ratio of the sum of QRS TVA from ECG leads with monophasic QRS complexes (i.e., monophasic TVA) to the entire sum of QRS TVA from all ECG leads (i.e., monophasic TVA plus multiphasic TVA). The TVA measurements used in the calculation of PMonoTVA are obtained from QRS complex waveforms (q/QS, r/R, s/S, r′/R′, and s′/S′) of the dominant QRS complex template of all individual leads of the 12-lead ECG (i.e., leads I, II, III, aVF, aVL, aVR, V1, V2, V3, V4, V5, and V6).

Percent monophasic amplitude (PMonoAmp), as used herein, refers to the percentage of QRS amplitude found on monophasic QRS complexes of the 12-lead ECG. PMonoAmp is calculated as the percentage ratio of the sum of QRS amplitude from ECG leads with monophasic QRS complexes (i.e., monophasic amplitude) to the entire sum of QRS amplitude from all ECG leads (i.e., monophasic amplitude plus multiphasic amplitude). Representative amplitude measurements are attained from QRS complex waveforms (q/QS, r/R, s/S, r′/R′, and s′/S′) of the dominant QRS complex template of individual leads of the 12-lead ECG (i.e., leads I, II, III, aVF, aVL, aVR, V1, V2, V3, V4, V5, and V6).

In some aspects, higher PMonoTVA and PMonoAmp values occurred among patients with VT as compared to patients with SWCT. The conceptual basis for these engineered features helping differentiate WCTs relates to the manner in which ventricular depolarization most commonly occurs for VT and SWCT. Without being limited to any particular theory, for many patients with VT, ventricular depolarization wavefronts primarily spread from their site-of-origin (SOO) using cardiomyocyte-to-cardiomyocyte conduction, with little-to-no assistance from the specialized conduction tissue. As a result, the ventricular depolarization wavefronts of VT tend to spread uniformly sans swift engagement and subdivision along an arborized His-Purkinje network that accounts for diametrically opposed and temporally separated QRS complex waveforms. Correspondingly, patients with VT will often exhibit monophasic QRS complexes (i.e., monophasic ‘QS complexes’ for ventricular depolarization wavefronts propagating away from any of the 12 ECG leads or monophasic ‘R complexes’ propagating toward any of the 12 ECG leads), especially if the SOO is remote from specialized conduction tissue or subjacent to the ventricular epicardium. Exemplary monophasic QRS waveforms. It is to be noted that monophasic QRS complex waveforms may possess positive or negative amplitudes with respect to the isoelectric baseline.

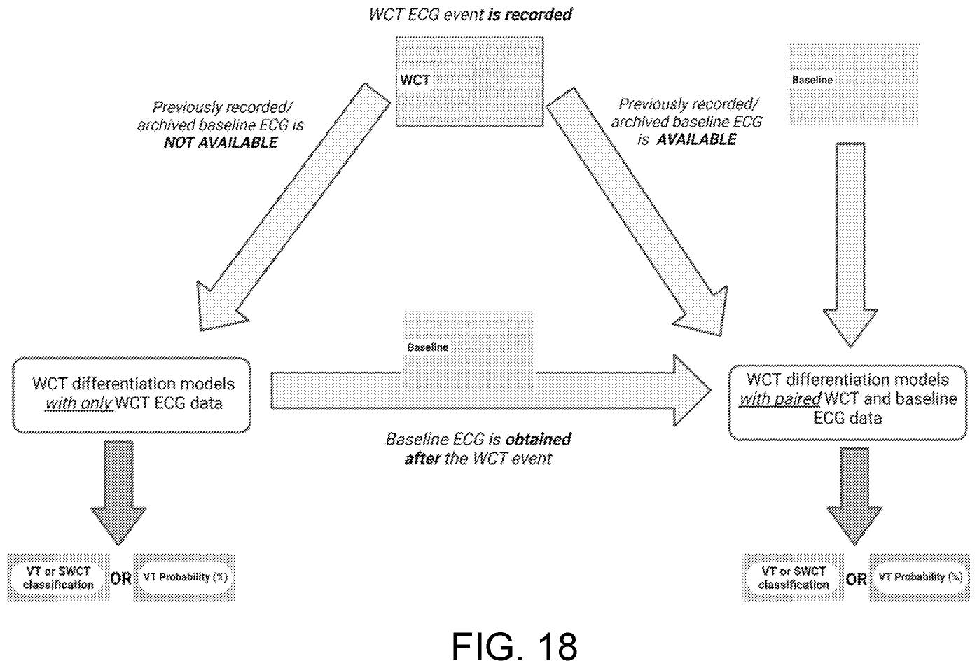

In contrast, most SWCT subtypes of WCT rely heavily upon the heart's specialized conduction tissue—not only as the primary link that couples supraventricular impulses to ventricular depolarization, but also as the indispensable conduit by which biventricular depolarization is accomplished. Among SWCTs arising from aberrant conduction (e.g., left bundle branch block (LBBB) or right bundle branch block (RBBB)), the rapid initial stages of ventricular depolarization affected by via specialized conduction tissue conduction are subsequently followed by a slower ventricular depolarization wavefront, which relies upon slower cardiomyocyte-to-cardiomyocyte conduction. As a result, the separate and distinct components of ventricular depolarization (i.e., initial vs. late) tend to produce multiphasic QRS complexes having diametrically opposed and temporally separated QRS complex waveform components (i.e., positive QRS waveforms [r/R, r′/R′] vs. negative QRS waveforms [q/Q, s/S, s′/S′]), shown illustrated schematically in FIG. 18.

In some aspects, the engineered features further include at least one of frontal percent amplitude change (Frontal PAC), horizontal percent amplitude change (Horizontal PAC), frontal percent time-voltage area change (Frontal PTVAC), and horizontal percent time-voltage area change (Horizontal PTVAC). Frontal and horizontal PAC and PTVAC are calculations that broadly quantify the extent of QRS amplitude change and TVA (time-voltage area) change, respectively, between WCT ECGs 105 and baseline ECGs 110.

Frontal and horizontal PACs are quantifiable measures of QRS amplitude changes between paired WCT and baseline ECG recordings. They are derived from computerized QRS waveform (q/QS, r/R, s/S, r′/R′, and s′/S′) amplitude (μV) measurements from corresponding ECG leads of the frontal (aVR, aVL, aVF) and horizontal (V1, V4, V6) ECG planes, respectively.

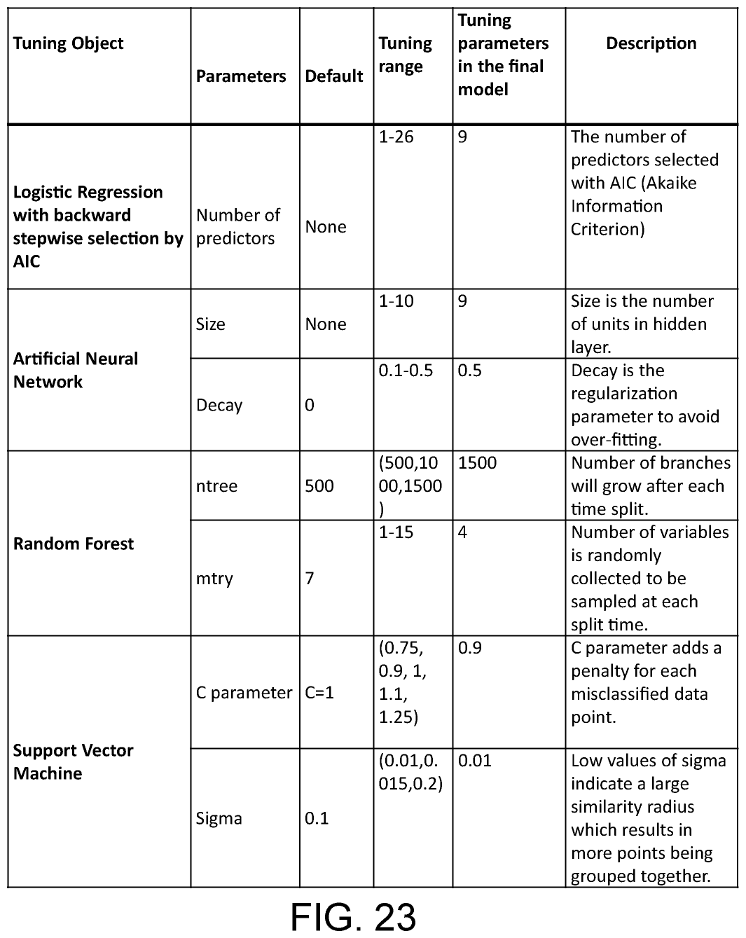

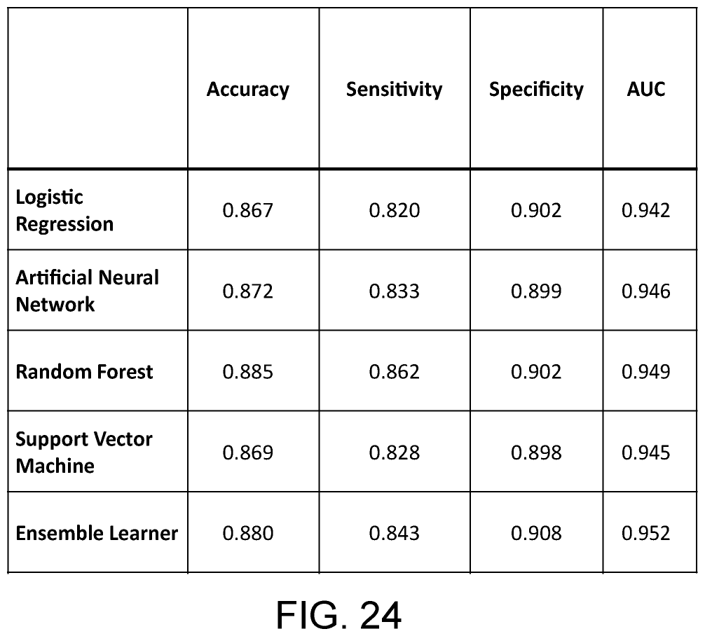

Frontal and horizontal PTVACs are quantifiable measures of the degree of change in QRS TVA (time-voltage area) between paired WCT and baseline ECGs. The processes of calculating the frontal and horizontal PTVACs are illustrated schematically in FIGS. 23 and 24, respectively. They are derived from QRS complex waveform (q/QS, r/R, s/S, r′/R′, and s′/S′) TVA measurements from specific ECG leads within the corresponding frontal (aVR, aVL, aVF) or horizontal (V1, V4, V6) ECG planes, respectively.

In some aspects, the engineered features further include at least one of WCT QRS duration, Baseline QRS Duration, and QRS duration changes. QRS duration, as used herein, refers to the total duration of the QRS waveform. WCT QRS duration and baseline QRS duration may be obtained from WCT and baseline ECG data, respectively using any suitable method without limitation including, but not limited to, manual ECG data analysis methods and automated ECG data analysis methods. In one aspect, WCT QRS duration and baseline QRS duration are obtained using any suitable automated data analysis software.

QRS duration change, as used herein, refers to the absolute difference in QRS duration (ms) measurements between paired WCT and baseline ECGs. In one aspect, QRS duration change is obtained by calculating the difference between the WCT QRS duration and the baseline QRS duration.

Without being limited to any particular theory, patients presenting with VT may exhibit greater WCT QRS duration, baseline QRS duration, and change in QRS duration than patients with SWCT. Engineered features relating to QRS duration (i.e., WCT QRS duration, baseline QRS duration, and absolute change in QRS duration between the WCT and baseline ECG) are useful in differentiating WCTs. WCTs having greater WCT QRS duration, baseline QRS duration, and absolute change in QRS duration are more likely secondary to VT, while WCTs demonstrating lesser values were more likely due to SWCT.

VT commonly expresses longer QRS durations than SWCT. Though VT and SWCT occupy broad and overlapping QRS duration ranges, the dissimilarity in QRS duration magnitude between these rhythms may be explained by differences in the manner by which VT and SWCT typically depolarize the ventricular myocardium. As noted herein, VT commonly relies upon a less efficient means of wavefront propagation (i.e., cardiomyocyte-to-cardiomyocyte) to depolarize the ventricular myocardium, which in turn tends to produce a prolonged QRS duration. In contrast, most SWCTs make use of the patient's native conduction system to rapidly accomplish the initial stages of ventricular depolarization, thereby reducing the amount of ventricular myocardium that must be depolarized by slower cardiomyocyte-to-cardiomyocyte conduction.

The differences in baseline QRS duration between VT and SWCT patients may be explained by the underlying myocardial substrate from which both heart rhythms commonly develop. Given that VT, by and large, emerges from patients who have underlying structural heart disease, patients who develop VT will tend to have a higher prevalence of underlying ventricular conduction abnormalities (e.g., bundle branch block) or baseline ventricular pacing from an implanted intracardiac device (ICD); both of which may be directly responsible for a longer baseline QRS duration. Furthermore, antiarrhythmic medications (e.g., sodium-channel blockers) known to cause baseline QRS duration prolongation are frequently provided to patients who have a history of VT.

The differences in the extent of QRS duration change upon WCT onset or offset between VT and SWCT are thought to be directly secondary to their differing means of ventricular depolarization. Given that VT commonly originates well outside the normal conduction system, it will often generate a large increase in QRS duration, especially among patients whose baseline heart rhythm exhibits rapid and efficient ventricular depolarization via a healthy His-Purkinje network. In contrast, many SWCTs appropriate the same conduction system pathways as the baseline heart rhythm and will express minimal changes to QRS duration. Only SWCTs due to ‘functional’ aberrancy or emerging preexcitation, which generate newfound ventricular depolarization delays, demonstrate large QRS duration changes between the WCT and baseline ECGs.

In some aspects, the engineered features further include at least one of QRS axis change and T wave axis change. QRS axis change, as used herein, refers to the absolute difference in orientation of the frontal plane QRS axis between paired WCT and baseline ECGs. T wave axis change, as used herein, refers to the absolute difference in the orientation of the frontal plane T wave axis between paired WCT and baseline ECGs.

The process 100 also includes transforming the engineered features into a classification 130 of the SWCT using a model 125 in various aspects. In various other aspects, the process 100 further includes transforming the engineered features obtained into an estimated probability 130 that the WCT is a VT or an SWCT using a model 125. Any suitable model 125 may be used to transform the engineered features without limitation. In some aspects, a machine learning model 125 is used to transform the engineered features into a classification or probability of VT or SWCT. Non-limiting examples of suitable machine learning models include genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In one exemplary method, a logistic regression model 125 is used to transform the engineered features into a classification or probability 130 of VT or SWCT.

In various aspects, the engineered parameters derived from WCT ECGs 105 or paired baseline 110 and WCT ECGs 105 are incorporated into one or more WCT formulas to calculate VT probability using a logistic regression model 125. In some aspects, logistic regression models 125 make use of WCT predictor values derived solely from WCT ECG data 105. In other aspects, additional logistic regression models 125 make use of WCT predictor values derived solely from WCT ECG data 105 as well as additional WCT predictor values derived from changes between baseline ECG data 110 and WCT ECG data 105.

In various aspects, the machine learning model 125 transforms engineering features derived from WCT ECG data 105 only, without the need for baseline ECG data 110. In various aspects, the machine learning model 125 is a binary outcome logistic regression model 125 that transforms selected independent WCT predictors into a classification 130 of VT or SWCT. Non-limiting examples of suitable independent WCT predictors include the engineered parameters PMonoTVA and WCT QRS Duration.

In some aspects, a logistic regression model 125 can be used to transform the selected engineered features into a probability P 130 of VT. Each WCT predictor (Xx) is assigned beta coefficients (Bx) according to their influence on the binary outcome (VT vs. non-VT). The “constant” term (BO) represents the y-intercept of the least-squares regression line. The discrete measured or calculated WCT predictor values can be incorporated into a WCT formula including, but not limited to a weighted sum formula.

By way of non-limiting example, a WCT Model 125 can make use of two highly predictive engineered features derived solely from the WCT ECG: (i) a standard and universally reported ECG measurement (i.e., WCT QRS duration) and (ii) computationally derived TVA measurements of QRS complex waveforms, PMonoTVA. The WCT Model 125 delivers accurate VT probability estimates 130 any time a WCT is recorded (i.e., ‘captured’) by a 12-lead ECG device 205 whether or not a baseline ECG 110 has been recorded.

In addition, the use of the WCT Model 125 (also known as the Solo Model) to differentiate WCT ECG data 105 can be coordinated with other high-performing models 125 that require computerized data from paired WCT 105 and baseline ECGs 110. For example, once a previously absent baseline ECG 110 is recorded after the WCT event 105, the WCT Model 125 can cede its application to a more robust prediction model that makes use of ECG data 105 provided by paired WCT 105 and baseline ECGs 110. However, if a patient already possesses a digitally archived baseline ECG 110 that is available for automated WCT differentiation algorithm application, VT or SWCT classification or VT probability estimation may be executed by more robust models 125 that leverage paired WCT 105 and baseline ECG 110 comparisons, and thereby supersede the predictions attained by the initial WCT Model 125, which only analyzes computerized ECG data 105 provided by the WCT itself.

The WCT differentiation methods disclosed herein transform 120 computerized ECG data, routinely processed by ECG interpretation software programs, into engineered parameters that are integrated into binary classification models 125 that use machine learning methods including, but not limited to logistic regression. An advantage of the disclosed WCT differentiation methods is that they provide clinicians with an impartial estimation of VT likelihood (i.e., 0.00% to 99.99% VT probability) using a procedure that functions independently of a clinician's ECG interpretation expertise or competency. Therefore, clinicians using the disclosed methods would be able to integrate automatically provided VT probability estimates 130 with the probabilistic influence of traditional WCT differentiation methods such as the Brugada algorithm, or other relevant clinical information such as a history of prior VT. In some aspects, the disclosed WCT differentiation methods are suitable for integration and/or implementation with commercially available ECG interpretation software platforms to more adeptly assist clinicians in distinguishing VT and SWCT accurately.

FIG. 2 depicts a simplified block diagram of an exemplary computer system 200 for implementing process 100 (shown in FIG. 1). In the exemplary embodiment, system 200 may be used for the computer-aided process 100 of classifying a wide complex tachycardia (WCT) pattern in a subject as described herein. The WCTA server 210 may be configured to implement at least a portion of the tasks associated with the disclosed methods of classifying the WCT patterns as described herein. As described below in more detail, a wide QRS complex tachycardia (WCT) Differentiation analysis (“WCTA”) server 210 may be configured to a) receive WCT electrocardiogram (ECG) data 105 indicative of a WCT pattern; b) transform 120 the WCT ECG data 105 into at least one engineering feature; c) execute at least one machine learning model 125 to analyze the at least one engineering feature and to output a classification 130 of the WCT pattern; d) based upon the classification 120 of the WCT pattern, determine whether the WCT pattern is indicative of at least one of a ventricular tachycardia (VT) and a supraventricular wide complex tachycardia (SWCT); and e) select a treatment 135 for the subject based upon the determination (all shown in FIG. 1).

In the exemplary embodiment, an electrocardiogram (ECG) device 205 measures voltage readings at different locations of a patient's body. The ECG device 205 generates EGC data 105 (shown in FIG. 1) including at least one summary parameter including, but not limited to, the durations and/or amplitudes characterizing a QRS complex waveform as described herein. Non-limiting examples of ECG devices 205 include, but are not limited to, a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), a pacemaker, an automatic implantable cardioverter defibrillator (AICD), an automated external defibrillator (AED), and any other suitable ECG measurement system or device 205. In the exemplary embodiment, the ECG device 205 is in communication with a user computer device 230 and/or a WCTA server 210, such as via the Internet or other computer network 225. More specifically, ECG devices 205 are communicatively coupled to the Internet through many interfaces including, but not limited to, at least one of a network 225, such as the Internet, a local area network (LAN), a wide area network (WAN), or an integrated services digital network (ISDN), a dial-up-connection, a digital subscriber line (DSL), a cellular phone connection, and a cable modem.

In the exemplary embodiment, user computer devices 230 are computers that include a web browser or a software application, which enables user computer devices 230 to access WCTA server 210 using the Internet. More specifically, user computer devices 230 are communicatively coupled to the Internet through many interfaces including, but not limited to, at least one of a network 225, such as the Internet, a local area network (LAN), a wide area network (WAN), or an integrated services digital network (ISDN), a dial-up-connection, a digital subscriber line (DSL), a cellular phone connection, and a cable modem. User computer devices 230 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices.

A database server 215 may be communicatively coupled to a database 220 that stores data. In one embodiment, database 220 may include ECG data 105, model data, baseline ECG data 110 (shown in FIG. 1, and/or user preferences. In the exemplary embodiment, database 220 may be stored remotely from WCTA server 210. In some embodiments, database 220 may be decentralized. In the exemplary embodiment, a person may access database 220 via user computer devices 230 by logging onto WCTA server 210, as described herein.

WCTA server 210 may be communicatively coupled with one or more user computer devices 230, ECG devices 205, and treatment devices 235. In the exemplary embodiment, WCTA server 210 are computers that include a web browser or a software application, which enables WCTA server 210 to access user computer devices 230, ECG devices 205, and treatment devices 235 using the Internet or other computer network 225. More specifically, WCTA server 210 is communicatively coupled to the Internet through many interfaces including, but not limited to, at least one of a network 225, such as the Internet, a local area network (LAN), a wide area network (WAN), or an integrated services digital network (ISDN), a dial-up-connection, a digital subscriber line (DSL), a cellular phone connection, and a cable modem. WCTA server 210 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices

One or more treatment devices 235 may be communicatively coupled with WCTA server 210 and/or user computer devices 230. The one or more treatment devices 235 each may be associated with a different diagnosis or treatment. More specifically, treatment devices 235 are communicatively coupled to the Internet through many interfaces including, but not limited to, at least one of a network 225, such as the Internet, a local area network (LAN), a wide area network (WAN), or an integrated services digital network (ISDN), a dial-up-connection, a digital subscriber line (DSL), a cellular phone connection, and a cable modem. Treatment devices 240 may be any device capable of accessing the Internet including, but not limited to, a mobile device, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), or XR (extended reality) headsets or glasses), chat bots, or other web-based connectable equipment or mobile devices.

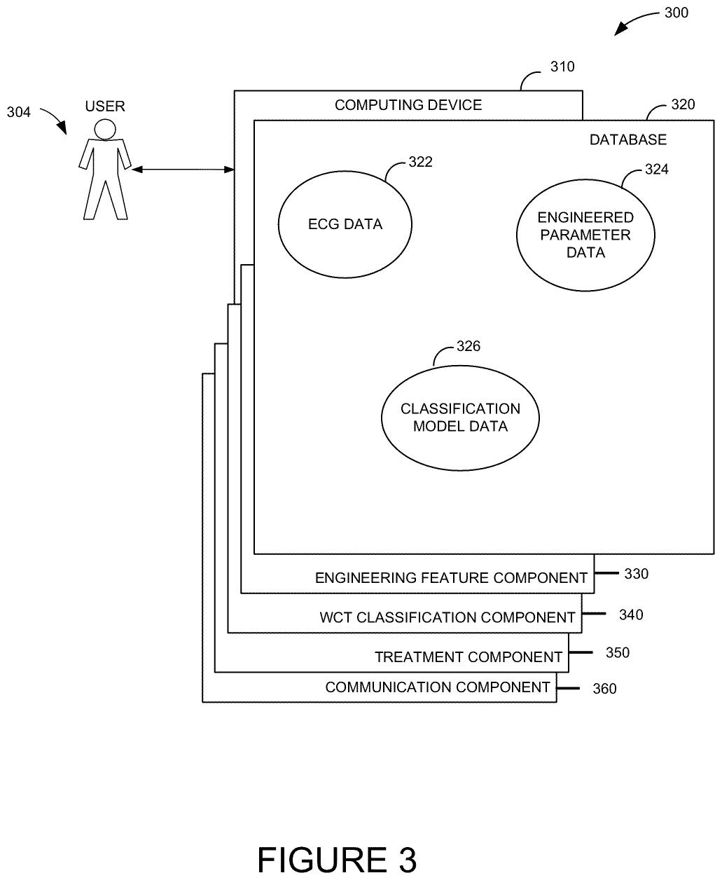

FIG. 3 illustrates a component configuration 300 of computing device 302, which includes database 310 along with other related computing components. In some aspects, computing device 302 is similar to WCTA server 210 (shown in FIG. 2). A user 304 may access components of computing device 302. In some aspects, database 310 is similar to database 220 (shown in FIG. 2).

In one aspect, database 310 includes ECG data 322, engineered parameter data 324, and classification model data 326. Non-limiting examples of suitable ECG data 322 include any values of parameters defining the baseline ECG data 110 and/or WCT ECG data 105 (both shown in FIG. 1) measured by the ECG device 205 (shown in FIG. 2). In some aspects, the ECG data 322 may include values of one or more engineered features computed by automated ECG data analysis software based on the ECG data 105 and 110 as described herein.

Non-limiting examples of engineered parameter data 324 include one or more parameters defining the transforms used to produce the various engineered features based on the ECG data 322 as described herein. By way of non-limiting example, the engineered parameter data 318 may include various values used to define the calculation of the engineered parameters Monophasic TVA, Multiphasic TVA, and PMonoTVA as described herein. In other aspects, the engineered parameter data 324 may include values of the engineered parameters calculated as described herein, for use in classifying the WCT ECG pattern as described herein.

Non-limiting examples of the classification model data 326 include values defining the architecture and specific implementations of one or more of the machine learning models 125 (shown in FIG. 1) used to classify the WCT ECG based on the engineered parameter values as described herein. In some aspects, the classification model data 326 may include training data used to train the machine learning models 125 as described herein. In additional aspects, the classification model data 326 may include the WCT classification produced by the machine learning model 125 as described herein, treatment recommendations based on the WCT classifications as described herein, and the treatment rules used to produce the treatment recommendations as described herein.

Computing device 302 also includes a number of components that perform specific tasks. In the exemplary aspect, computing device 302 includes engineered feature component 330, WCT classification component 340, treatment component 350, and communication component 360.

In various aspects, the engineered feature component 330 implements the transformation of the WCT ECG data 105 and/or baseline ECG data 110 into the various engineered features as described herein. In some aspects, the engineered feature component 330 receives at least a portion of the engineered parameters from automated data analysis software implemented using the disclosed system 200 or by an additional device including, but not limited to the ECG device 205 (shown in FIG. 2). In various aspects, the WCT classification component 340 implements the training of the machine learning model 125 and/or the classification 130 of the WCT ECG pattern as a VT or an SWCT using the trained machine learning model 125 as described herein. In various aspects, the treatment component 350 implements the transformation of the classification 130 of the WCT ECG pattern into a treatment recommendation 135 (shown in FIG. 1) as described herein. In some aspects, the treatment component 350 may produce signals used to operate a treatment device 235 (shown in FIG. 2).

Communication component 360 is configured to enable communications between computing device 402 and other devices (e.g. user computing device 230 shown in FIG. 2) over a network, such as network 225 (shown in FIG. 2), or a plurality of network connections using predefined network protocols such as TCP/IP (Transmission Control Protocol/Internet Protocol).

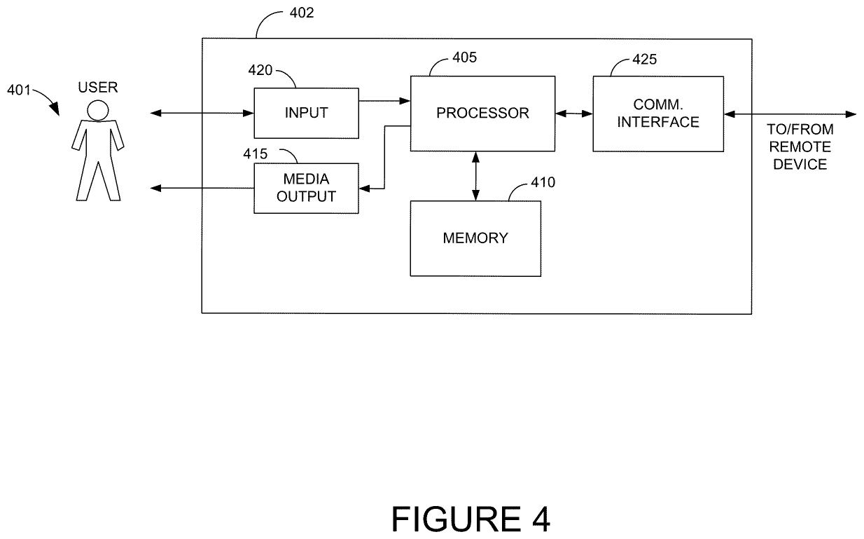

FIG. 4 depicts an exemplary configuration of a user computer device 230 (shown in FIG. 2), in accordance with one embodiment of the present disclosure. User computer device 402 may be operated by a user 401. User computer device 402 may include, but is not limited to, user computer devices 230, and treatment devices 235 (all shown in FIG. 2). User computer device 402 may include a processor 405 for executing instructions. In some embodiments, executable instructions are stored in a memory area 410. Processor 405 may include one or more processing units (e.g., in a multi-core configuration). Memory area 410 may be any device allowing information such as executable instructions and/or transaction data to be stored and retrieved. Memory area 410 may include one or more computer readable media.

User computer device 402 may also include at least one media output component 415 for presenting information to user 401. Media output component 415 may be any component capable of conveying information to user 401. In some embodiments, media output component 415 may include an output adapter (not shown) such as a video adapter and/or an audio adapter. An output adapter may be operatively coupled to processor 405 and operatively coupleable to an output device such as a display device (e.g., a cathode ray tube (CRT), liquid crystal display (LCD), light emitting diode (LED) display, or “electronic ink” display), an audio output device (e.g., a speaker or headphones), virtual headsets (e.g., AR (Augmented Reality), VR (Virtual Reality), or XR (extended Reality) headsets).

In some embodiments, media output component 415 may be configured to present a graphical user interface (e.g., a web browser and/or a client application) to user 401. A graphical user interface may include, for example, an online interface for viewing ECG analysis results. In some embodiments, user computer device 402 may include an input device 420 for receiving input from user 401. User 401 may use input device 420 to, without limitation, select and/or enter one or more ECG scans 105 (shown in FIG. 1) to analyze.

Input device 420 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 415 and input device 420.

User computer device 402 may also include a communication interface 425, communicatively coupled to a remote device such as the WCTA server 210 (shown in FIG. 2). Communication interface 425 may include, for example, a wired or wireless network adapter and/or a wireless data transceiver for use with a mobile telecommunications network.

Stored in memory area 410 are, for example, computer readable instructions for providing a user interface to user 401 via media output component 415 and, optionally, receiving and processing input from input device 420. A user interface may include, among other possibilities, a web browser and/or a client application. Web browsers enable users, such as user 401, to display and interact with media and other information typically embedded on a web page or a website from the WCTA server 210. A client application allows user 401 to interact with, for example, the WCTA server 210. For example, instructions may be stored by a cloud service, and the output of the execution of the instructions sent to the media output component 415.

Processor 405 executes computer-executable instructions for implementing aspects of the disclosure. In some embodiments, the processor 405 is transformed into a special purpose microprocessor by executing computer-executable instructions or by otherwise being programmed.

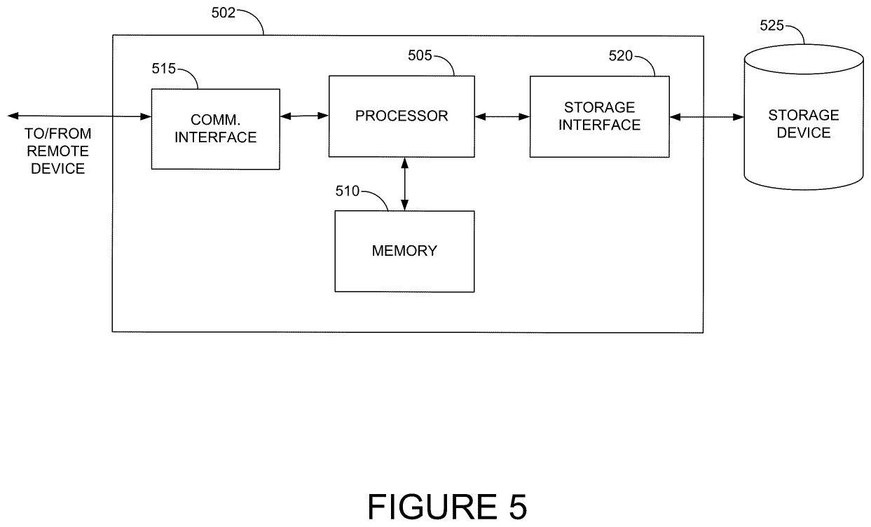

FIG. 5 depicts an exemplary configuration of a server 210 shown in FIG. 2, in accordance with one embodiment of the present disclosure. Server computer device 501 may include, but is not limited to, database server 215 and WCTA server 210 (both shown in FIG. 2). Server computer device 501 may also include a processor 505 for executing instructions. Instructions may be stored in a memory area 510. Processor 505 may include one or more processing units (e.g., in a multi-core configuration).

Processor 505 may be operatively coupled to a communication interface 515 such that server computer device 501 is capable of communicating with a remote device such as another server computer device 501, ECG device 205, or user computer devices 230 (both shown in FIG. 2). For example, communication interface 515 may receive requests from user computer devices 230 via the Internet, as illustrated in FIG. 2.

Processor 505 may also be operatively coupled to a storage device 534. Storage device 534 may be any computer-operated hardware suitable for storing and/or retrieving data, such as, but not limited to, data associated with database 220 (shown in FIG. 2). In some embodiments, storage device 534 may be integrated in server computer device 501. For example, server computer device 501 may include one or more hard disk drives as storage device 534.

In other embodiments, storage device 534 may be external to server computer device 501 and may be accessed by a plurality of server computer devices 501. For example, storage device 534 may include a storage area network (SAN), a network attached storage (NAS) system, and/or multiple storage units such as hard disks and/or solid state disks in a redundant array of inexpensive disks (RAID) configuration.

In some embodiments, processor 505 may be operatively coupled to storage device 534 via a storage interface 520. Storage interface 520 may be any component capable of providing processor 505 with access to storage device 534. Storage interface 520 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 505 with access to storage device 534.

Processor 505 may execute computer-executable instructions for implementing aspects of the disclosure. In some embodiments, the processor 505 may be transformed into a special purpose microprocessor by executing computer-executable instructions or by otherwise being programmed. For example, the processor 505 may be programmed with the instructions such as illustrated in FIG. 1.

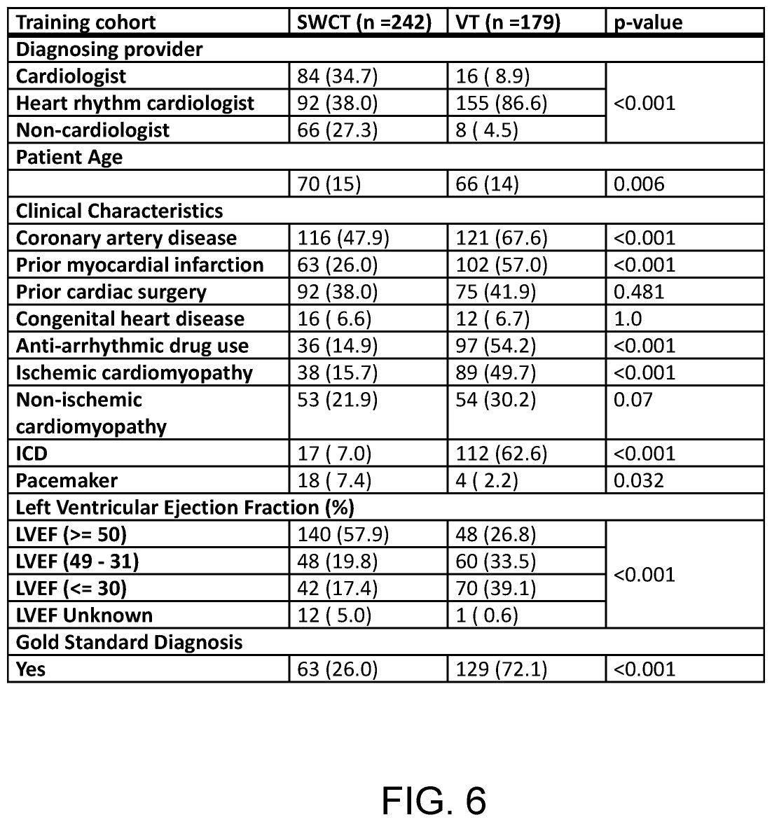

FIG. 6 illustrates an exemplary table describing the characteristics of the training cohort from Institution 1. The numbers in parentheses are percentages of n or standard deviation. Patients having a gold standard diagnosis were those with a corroborating EP or implantable intracardiac device recordings.

FIG. 7 illustrates an exemplary table describing the characteristics of the training cohort from Institution 2. The numbers in parentheses are percentages of n or standard deviation. Patients having a gold standard diagnosis were those with a corroborating EP or implantable intracardiac device recordings.

FIG. 8 illustrates an exemplary table summarizing the diagnostic performance of various ML model subtypes in Part 1 (using 14 features) on gold standard and non-gold standard cohorts of the testing cohort (from Institution #2).

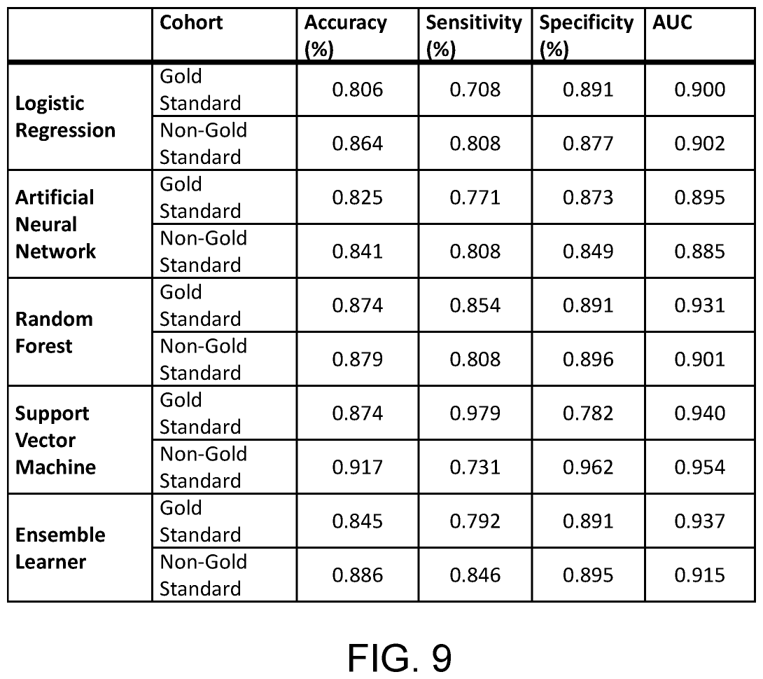

FIG. 9 illustrates an exemplary table summarizing the diagnostic performance of various ML model subtypes in Part 2 (using 14 features) on gold standard and non-gold standard cohorts of the testing cohort (from Institution #2).

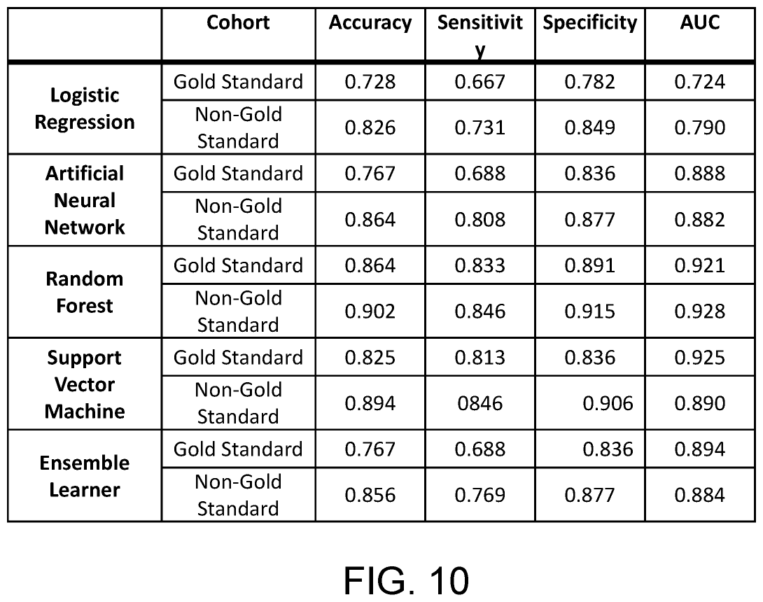

FIG. 10 illustrates an exemplary table summarizing the diagnostic performance of various ML model subtypes in Part 3 (using 26 features) on gold standard and non-gold standard cohorts of the testing cohort (from Institution #2).

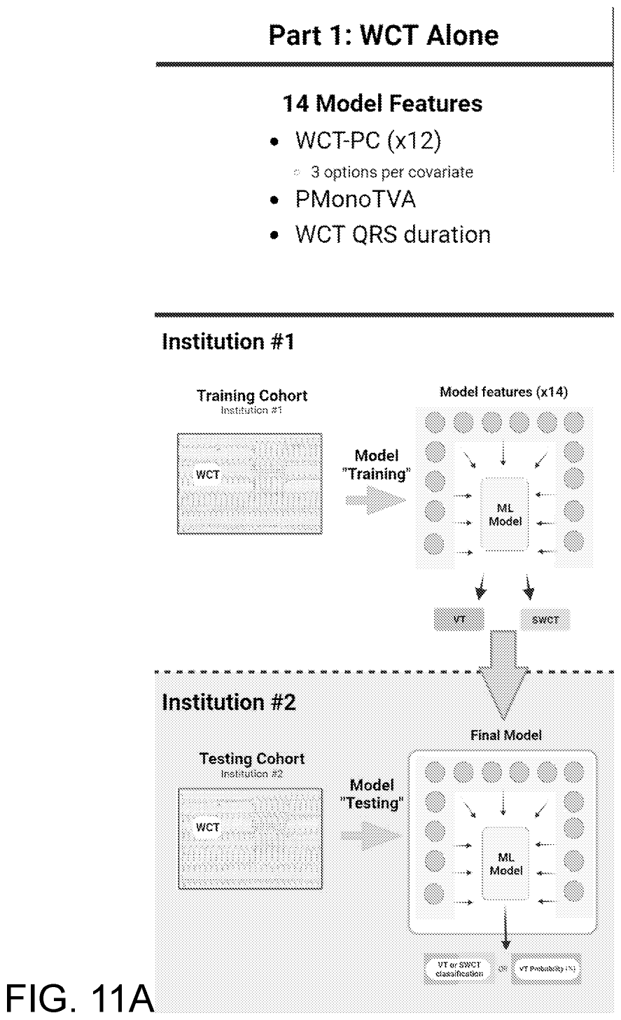

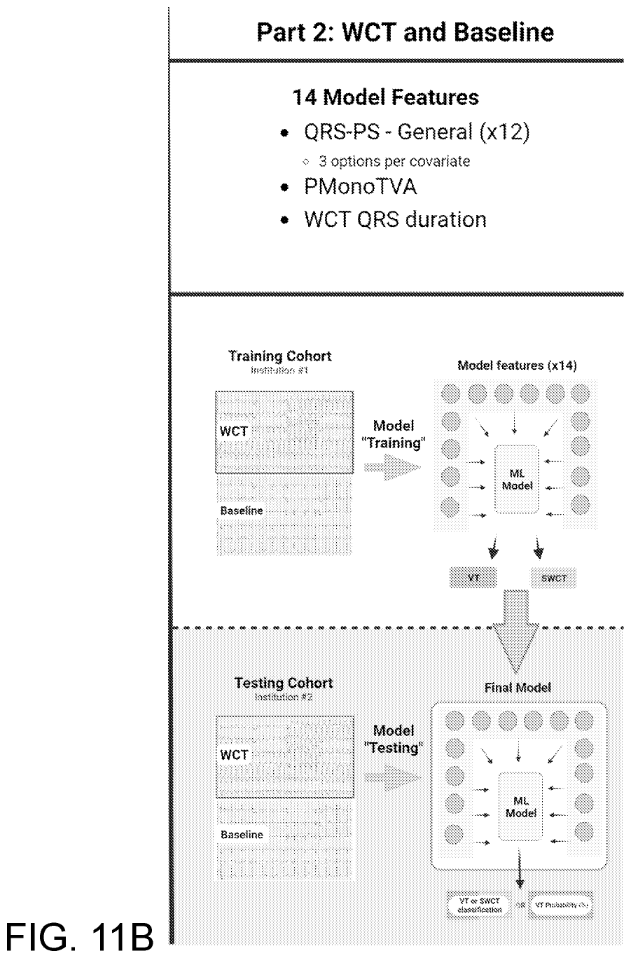

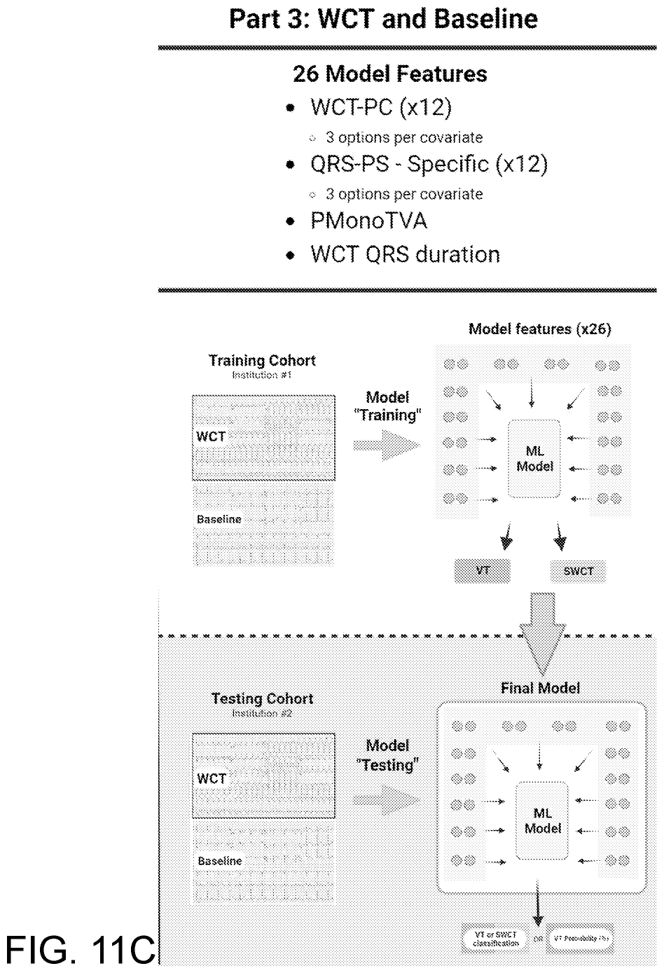

FIGS. 11A-11C illustrates an exemplary schematic of the three-part study design described in the current disclosure. Each part used a different set of model features to be incorporated into ML learning models. Model training used ECGs acquired at Institution #1. Model testing used ECGs acquired at Institution #2.

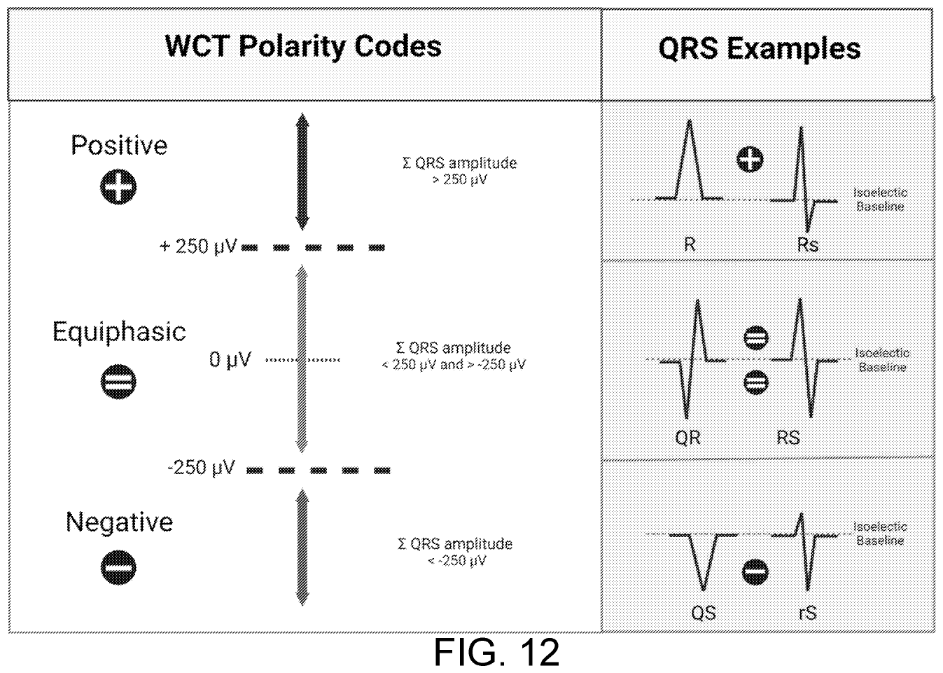

FIG. 12 illustrates an exemplary schematic describing how the WCT Polarity Codes were determined by way of summating QRS complex waveform amplitudes (μV) above (r/R and r′/R′) and below (q/QS, s/S, and s′/S′) the isoelectric baseline. For each calculation, the amplitude (μV) of QRS complex waveforms having a downward orientation (i.e., q/QS, s/S, and s′/S′) were considered numerically negative, while the amplitude (μV) of QRS complex waveforms having an upright orientation (i.e., r/R and r′/R′) were considered numerically positive. If the sum of (Σ) QRS complex waveforms (i.e., q/QS+r/R+s/S+r′/R′+s′/S′) was greater than 250 μV above the isoelectric baseline (i.e., Σ QRS amplitude >250 μV), the QRS complex is defined as having positive polarity. If the sum of QRS complex waveforms was more than 250 μV beneath the isoelectric baseline (i.e., Σ QRS amplitude <−250 μV), the QRS complex is defined as having negative polarity. If the sum of QRS complex waveforms falls between 250 μV above or beneath the isoelectric baseline (i.e., 250 μV<=∈QRS amplitude=>−250 μV), the QRS complex is defined as having equiphasic polarity.

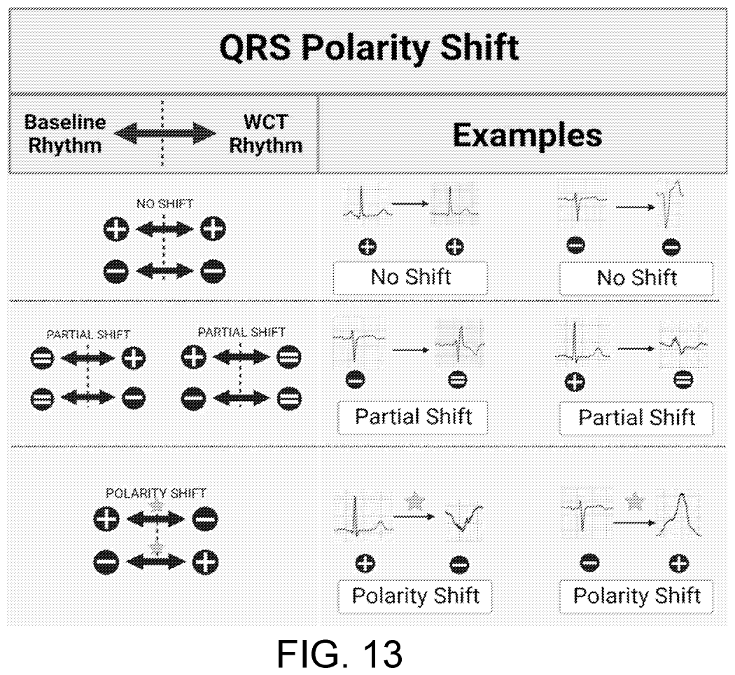

FIG. 13 illustrates an exemplary schematic showing examples of QRS polarity shift possibilities during the transition of a baseline ECG into a WCT ECG, and vice versa.

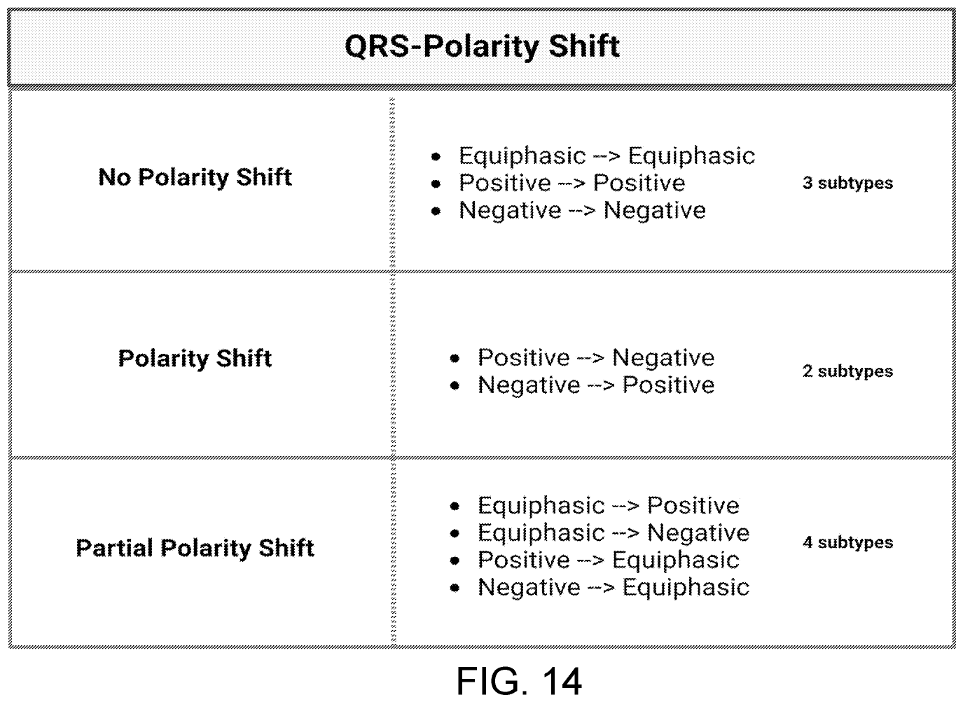

FIG. 14 illustrates an exemplary table showing categorical subtypes for QRS-Polarity Shift, including 3 general subtypes ([i] no polarity shift, [ii] polarity shift, and [iii] partial polarity shift) and 9 more specific subtypes ([i] equiphasic (=)→equiphasic (=), [ii] positive (+)→positive (+), [iii] negative (−)→negative (−), [iv] positive (+)→negative (−), [v] positive (+)→equiphasic (=), [vi] negative (−)→positive (+), [vii] negative (−)→equiphasic (=), [viii] equiphasic (=)→positive (+), and [ix] equiphasic (=)→negative (−)).

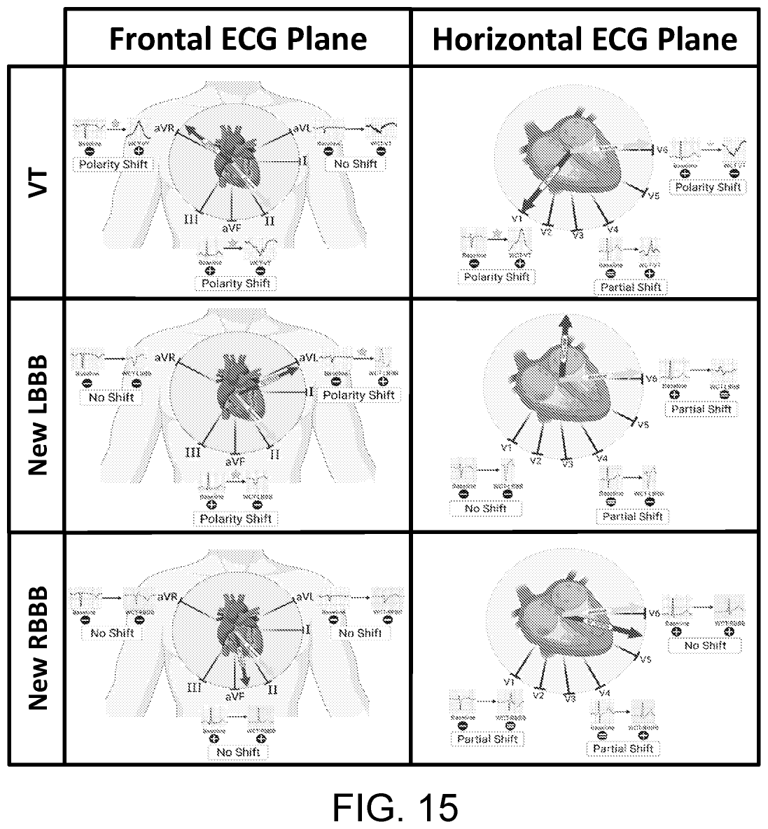

FIG. 15 illustrates an exemplary set of schematics showing examples of QRS Polarity Shift (QRS-PS) among examples of VT and SWCT. The changes in mean electrical vector from a baseline ECG into (i) VT, (ii) SWCT due to LBBB, and (iii) SWCT due to RBBB are demonstrated in the frontal (left panels) and horizontal plane (right panels). The polarity of QRS complexes is defined as positive, negative, or equiphasic based upon the directionality of the QRS complex waveform. Colored arrows represent the mean electrical vector of the WCT (red) and baseline (yellow) rhythms. Compared to new RBBB or LBBB, VT has multiple polarity shifts across the ECG leads shown. LBBB, left bundle branch block; RBBB, right bundle branch block; VT, ventricular tachycardia; WCT, wide QRS complex tachycardia.

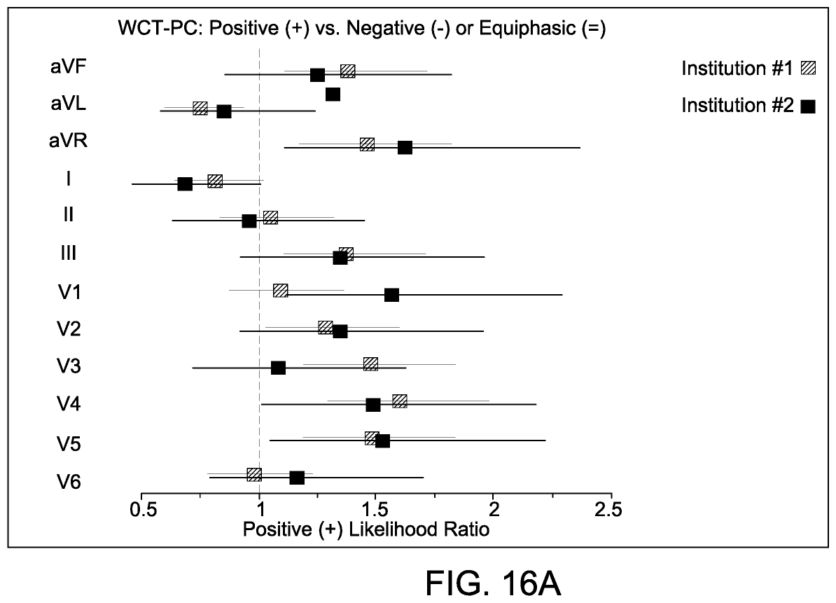

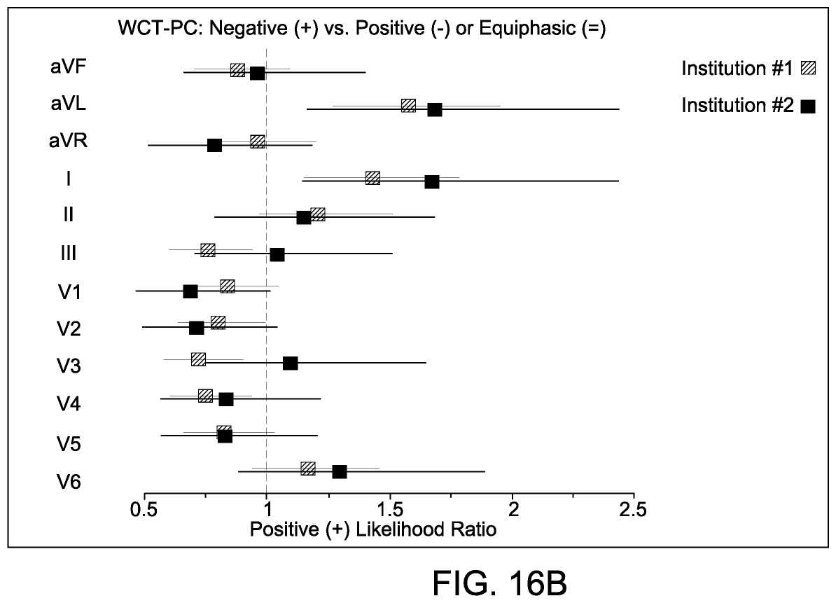

FIGS. 16A and 16B illustrates an exemplary pair of forest plots demonstrating the discriminatory capacity of positive and negative WCT-PCs among individual ECG leads. Plot squares and intervals represent the mean and confidence intervals, respectively (Institution #1, Institution #2). Upper panel: plot demonstrating positive likelihood ratios among ECG leads demonstrating a positive WCT-PC, as compared to negative or equiphasic WCT-PCs. Lower panel: plot demonstrating positive likelihood ratios among ECG leads demonstrating a negative (−) WCT-PC, as compared to positive (+) or equiphasic (=) WCT-PCs. Abbreviations: WCT-PC, WCT Polarity code.

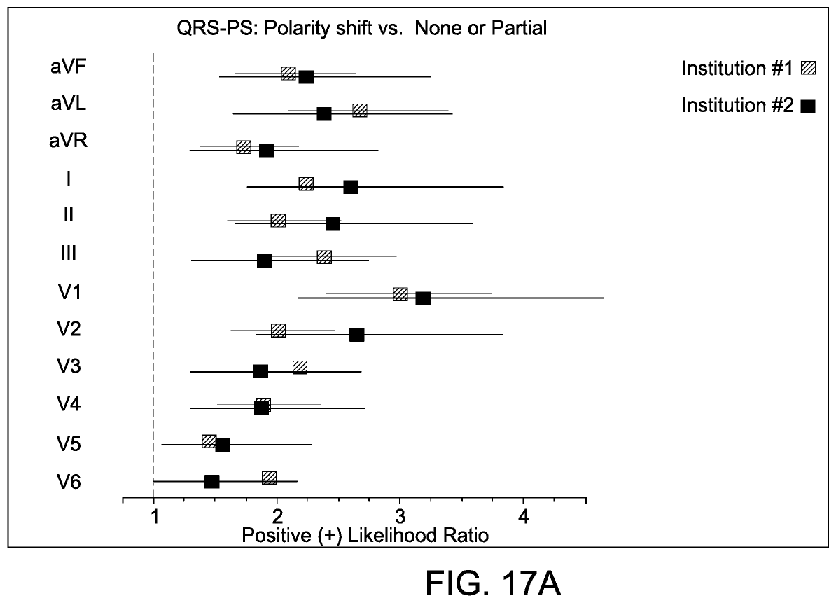

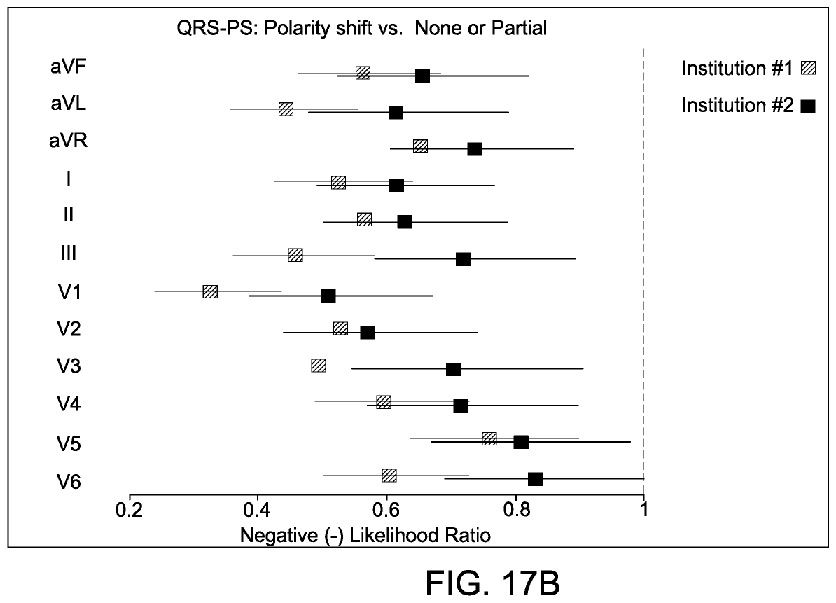

FIGS. 17A and 17B illustrates an exemplary pair of forest plots demonstrating the discriminatory capacity QRS-PS among individual ECG leads. Plot squares and intervals represent the mean and confidence intervals, respectively (Institution #1, Institution #2). Upper panel: plot demonstrating positive likelihood ratios among ECG leads demonstrating a polarity shift, as compared to a partial or no polarity shift. Lower panel: plot demonstrating negative likelihood ratios among ECG leads demonstrating a polarity shift, as compared to a partial or no polarity shift.

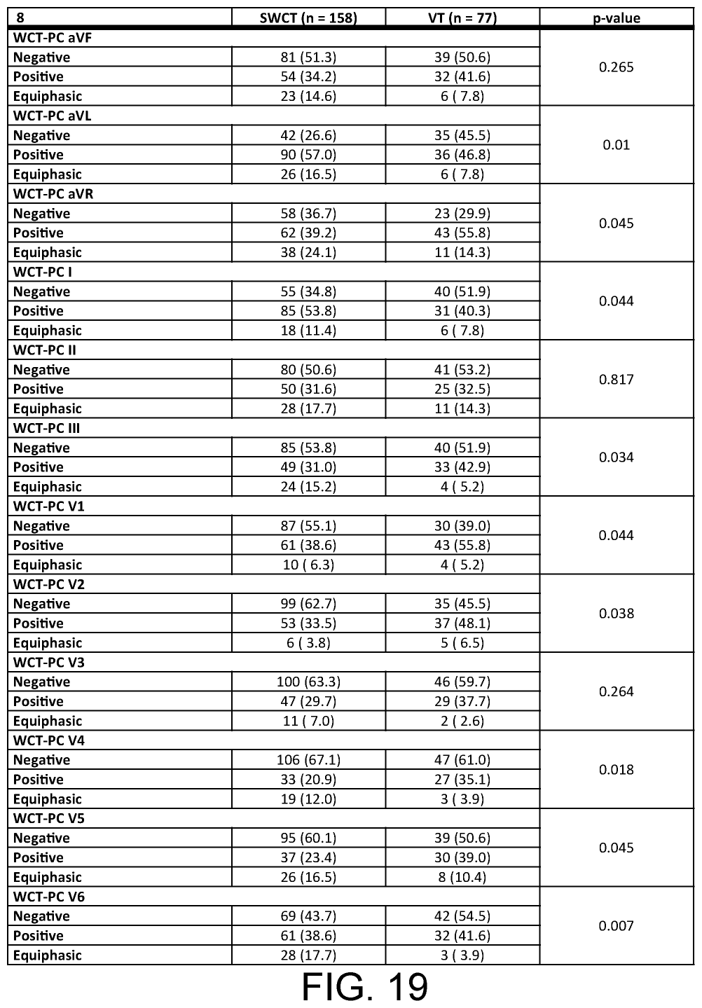

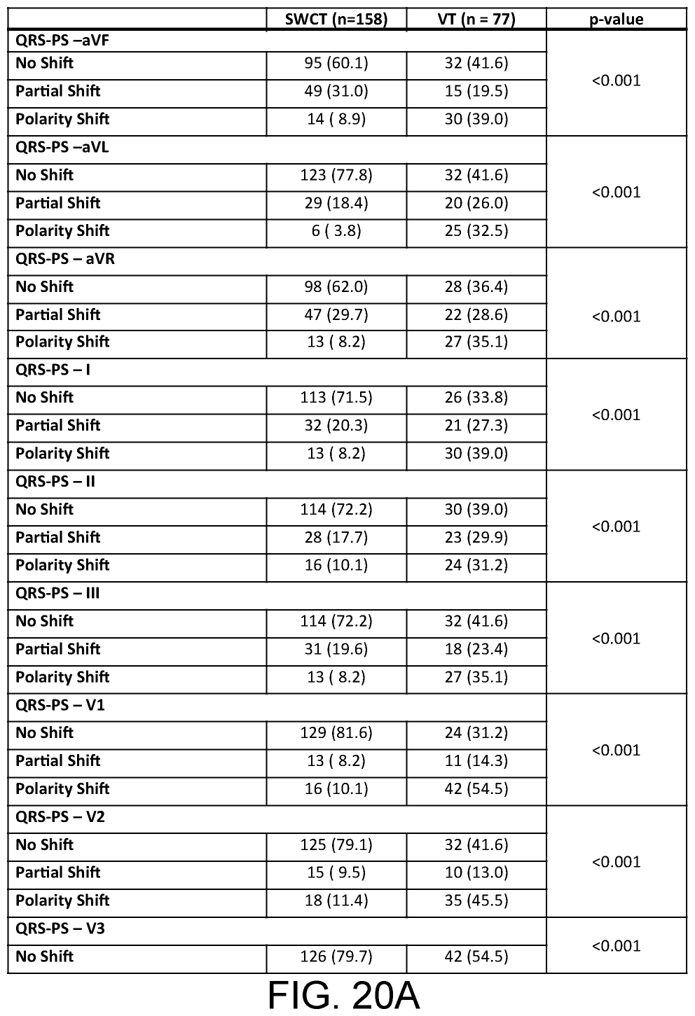

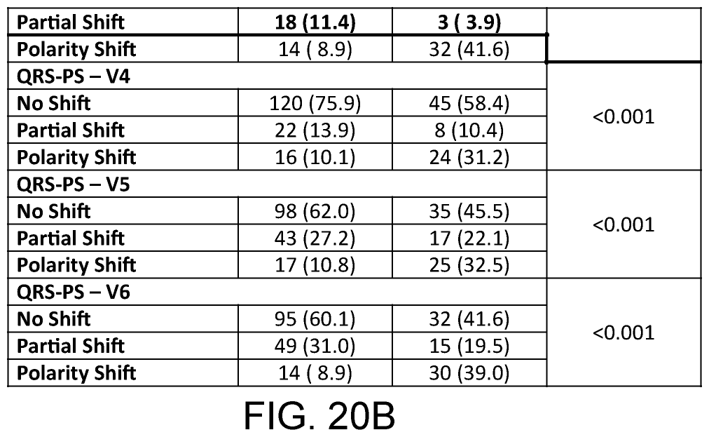

FIG. 18 illustrates an exemplary schematic of the proposed process for the WCT differentiation model application as determined by the presence or absence of a baseline ECG. FIG. 19 illustrates an exemplary table summarizing the ability of the models of the current disclosure to differentiate between SWCT and VT. FIGS. 20A and 20B illustrates an exemplary table showing frequency of QRS-PS among 12-lead ECG leads categorized according to 9 specific subtypes (WCT ECG→Baseline ECG): (i) equiphasic (=)→equiphasic (=), (ii) positive (+)→positive (+), (iii) negative (−)→negative (−), (iv) positive (+)→negative (−), (v) positive (+)→equiphasic (=), (vi) negative (−)→positive (+), (vii) negative (−)→equiphasic (=), (viii) equiphasic (=)→positive (+), and (ix) equiphasic (=)→negative (−).

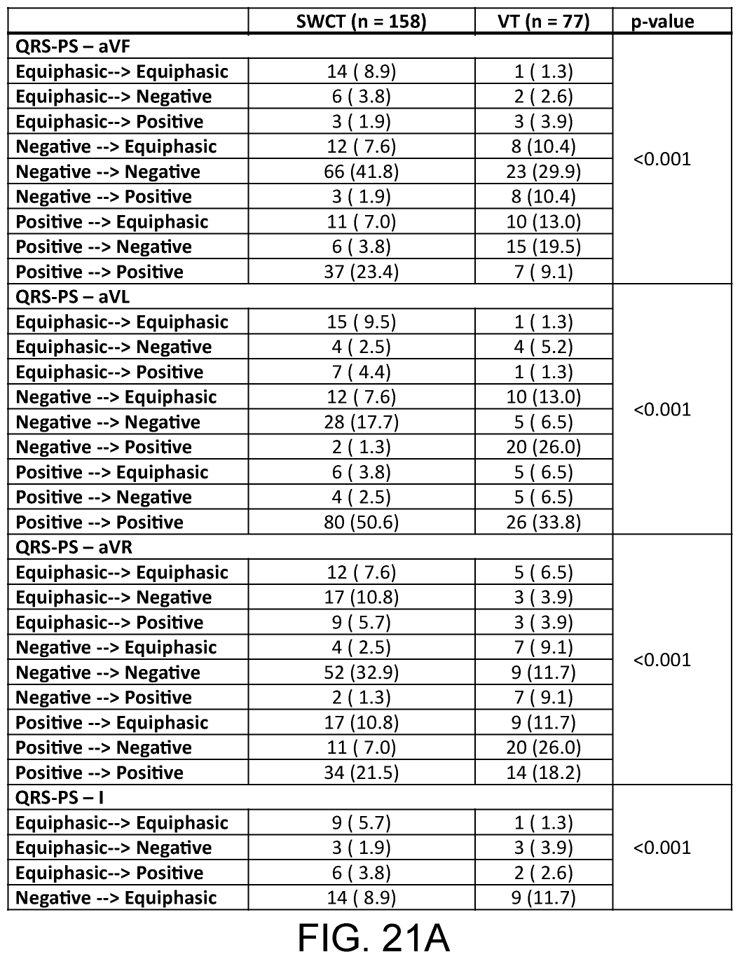

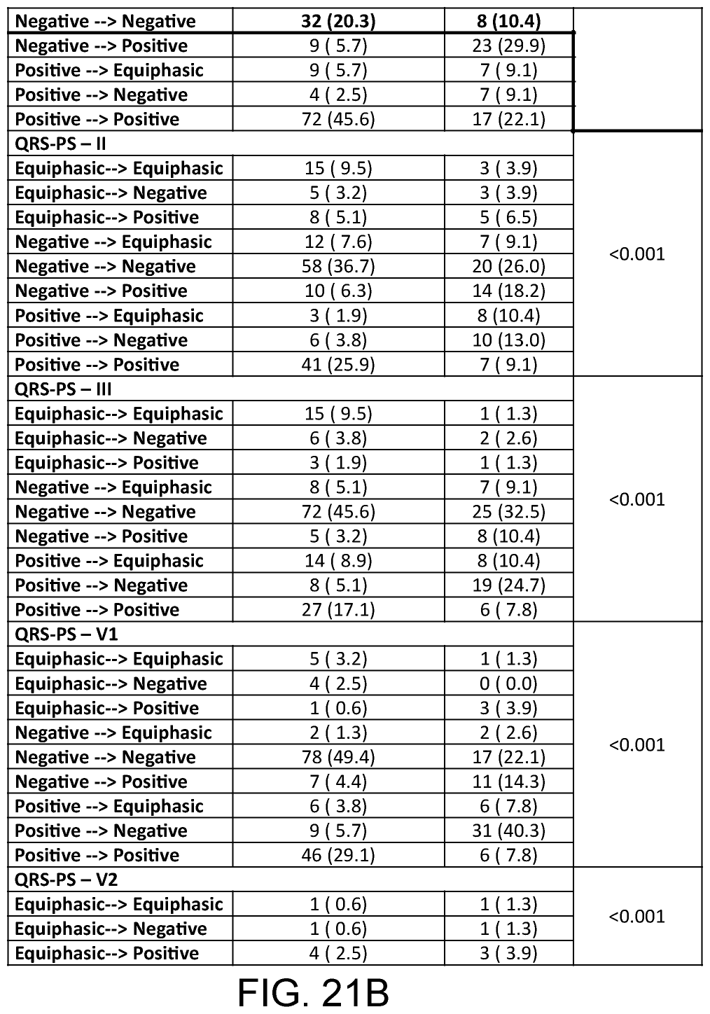

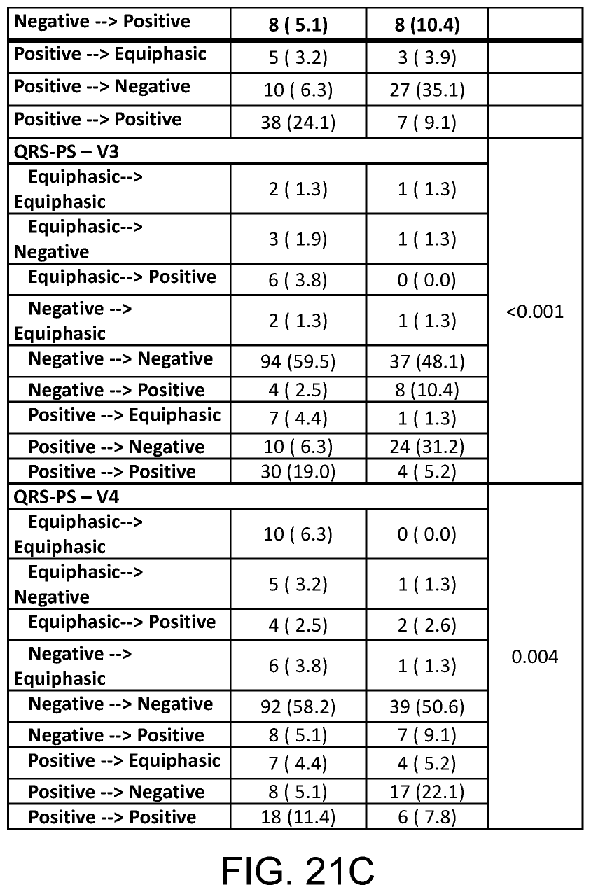

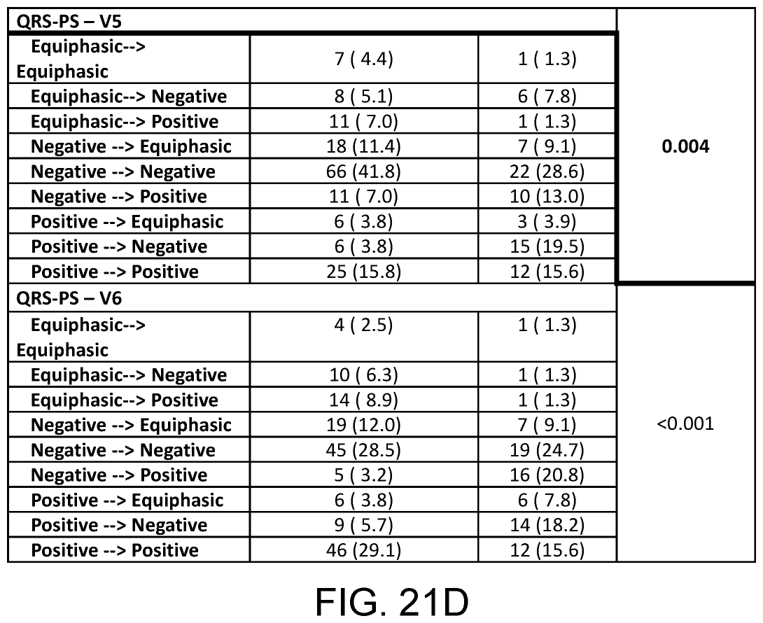

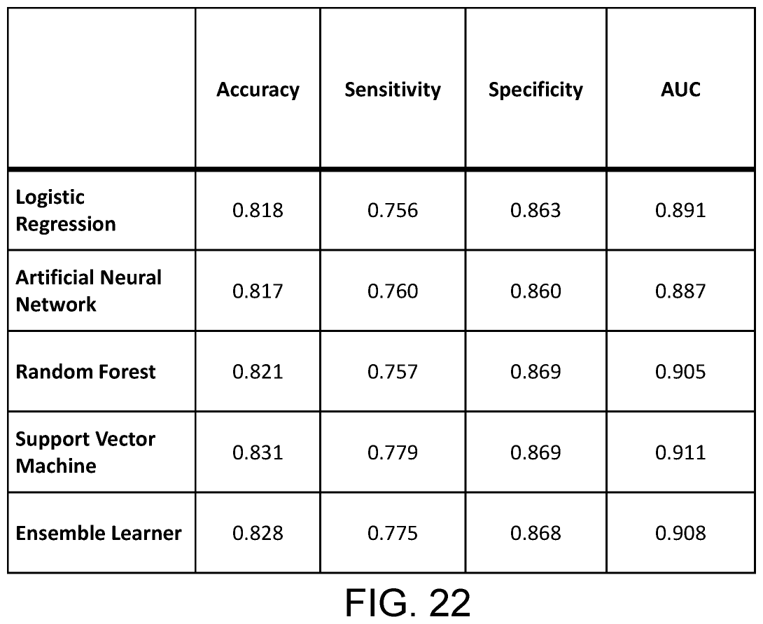

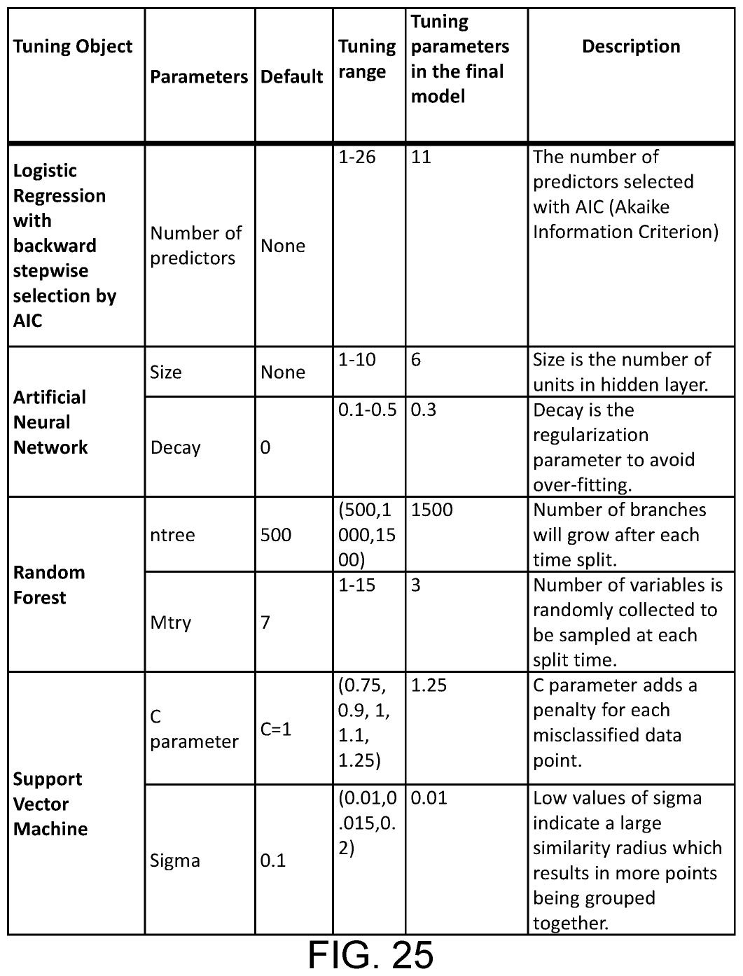

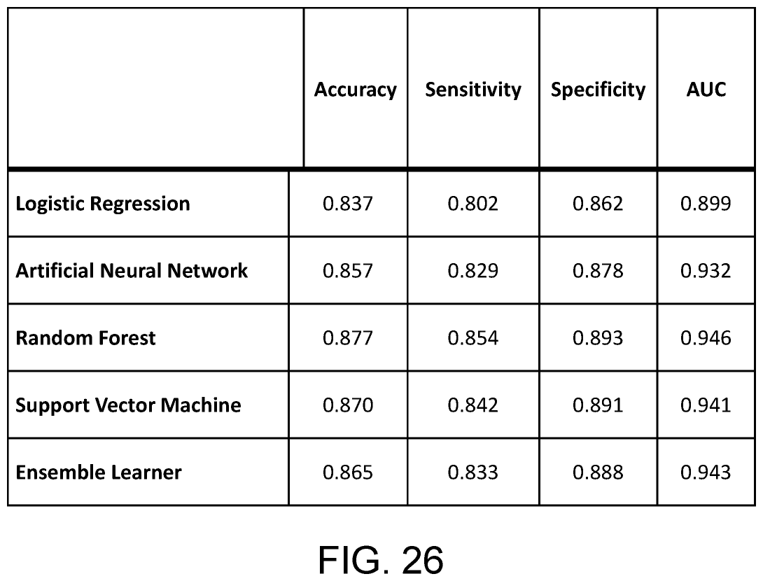

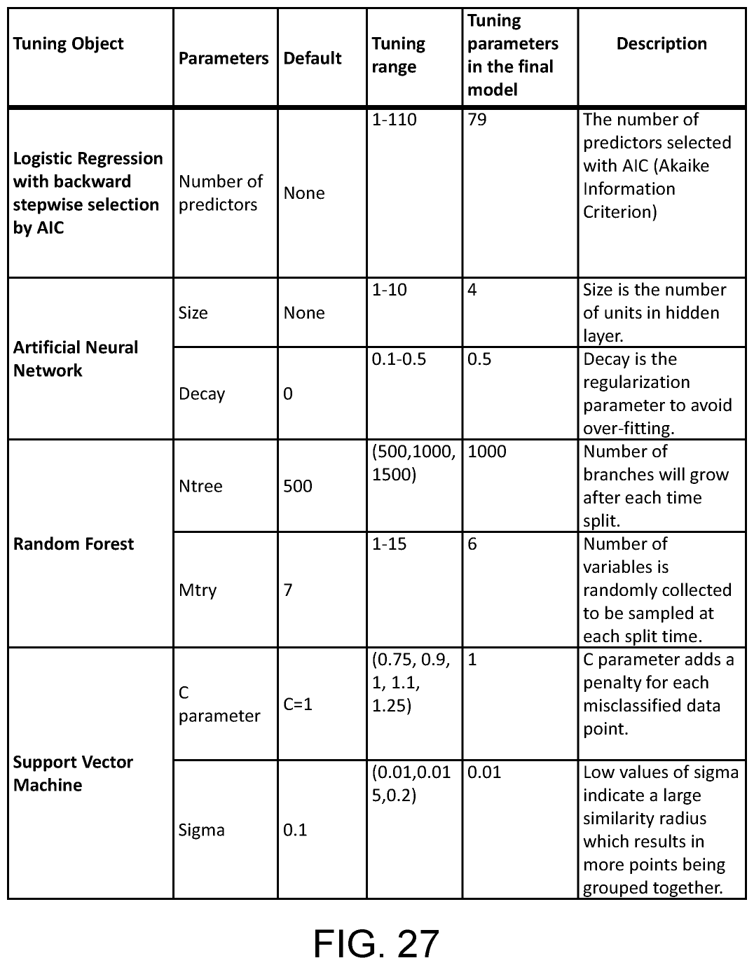

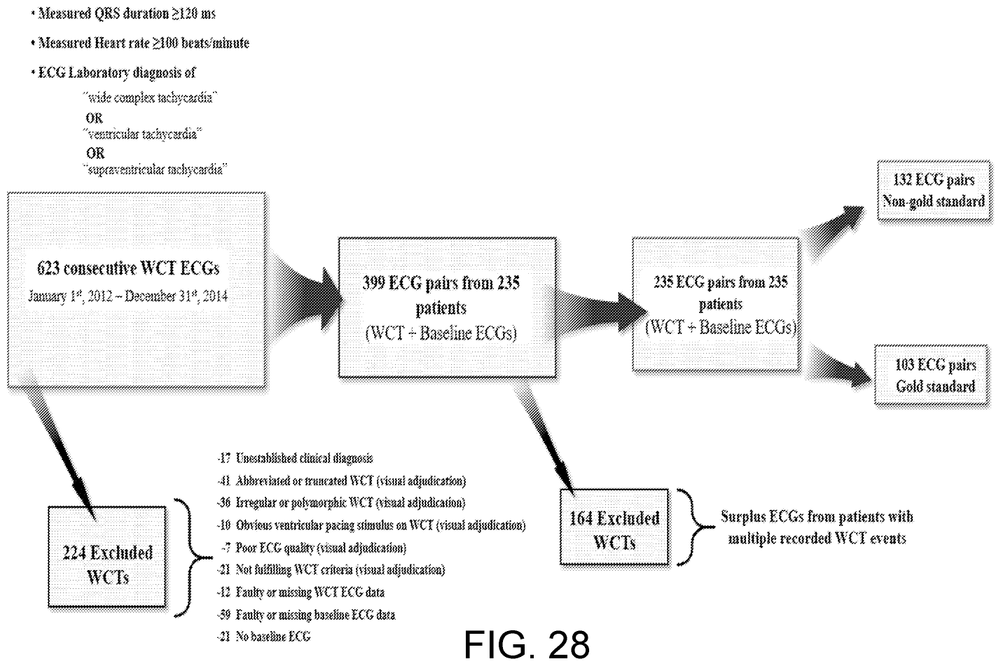

FIGS. 21A-21D illustrates an exemplary table showing the frequency of QRS-PS among the leads of the 12-lead ECG categorized into three general subtypes: [i] no polarity shift, [ii] polarity shift, and [iii] partial polarity shift. FIG. 22 illustrates an exemplary table summarizing the diagnostic performance of various ML model subtypes for Study Part 1. FIG. 23 illustrates an exemplary table summarizing the tuning parameters for ML model subtypes in Study Part 1 of the present disclosure. FIG. 24 illustrates an exemplary table summarizing the diagnostic performance of various ML model subtypes in Study Part 2 of the present disclosure. FIG. 25 illustrates an exemplary table summarizing the tuning parameters for ML model subtypes in Study Part 2. FIG. 26 illustrates an exemplary table summarizing the diagnostic performance of ML Model Training in Study Part 3. FIG. 27 illustrates an exemplary table summarizing the tuning parameters for ML model subtypes in Study Part 3. FIG. 28 illustrates an exemplary flow diagram depicting WCT ECG cohort selection (Institution #2).

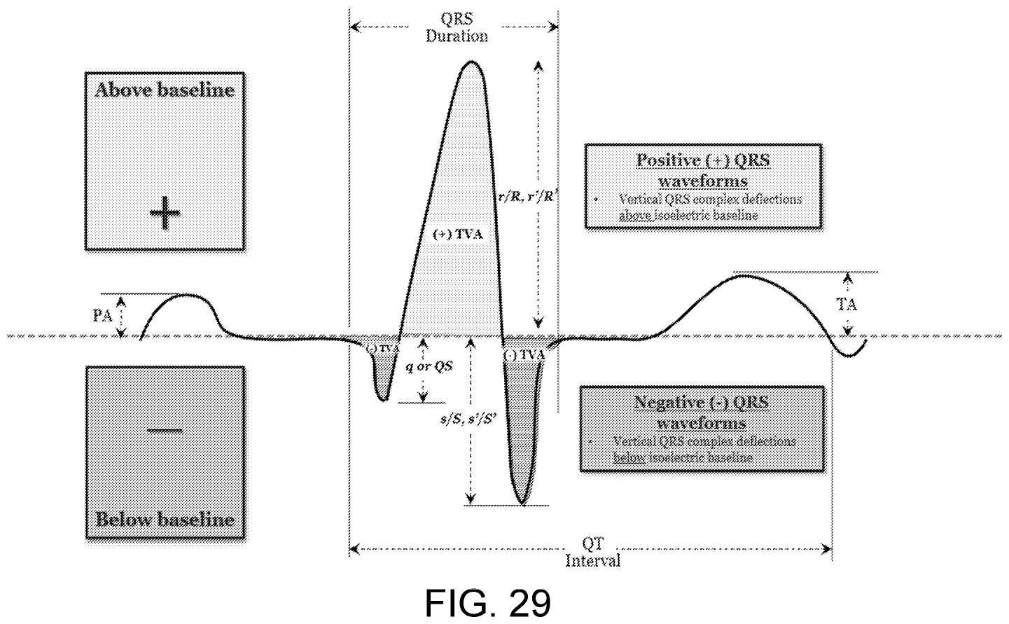

FIG. 29 illustrates an exemplary is a schematic of measurements of QRS complex waveforms showing a schematic representation of a QRS complex and its measurable components provided by computerized ECG interpretation software. QRS amplitude (μV) represents the vertical height of positive (r/R and r′/R′) and negative (q/QS, s/S, and s′/S′) QRS waveforms. QRS time-voltage areas (TVAs) (μV·ms) represent the “area” enveloped by individual QRS complex waveforms above (r/R and r′/R′) or below (q/QS, s/S, and s′/S′) the isoelectric baseline. Abbreviations: PA, P—wave amplitude; TA, T—wave amplitude; TVA, time-voltage area.

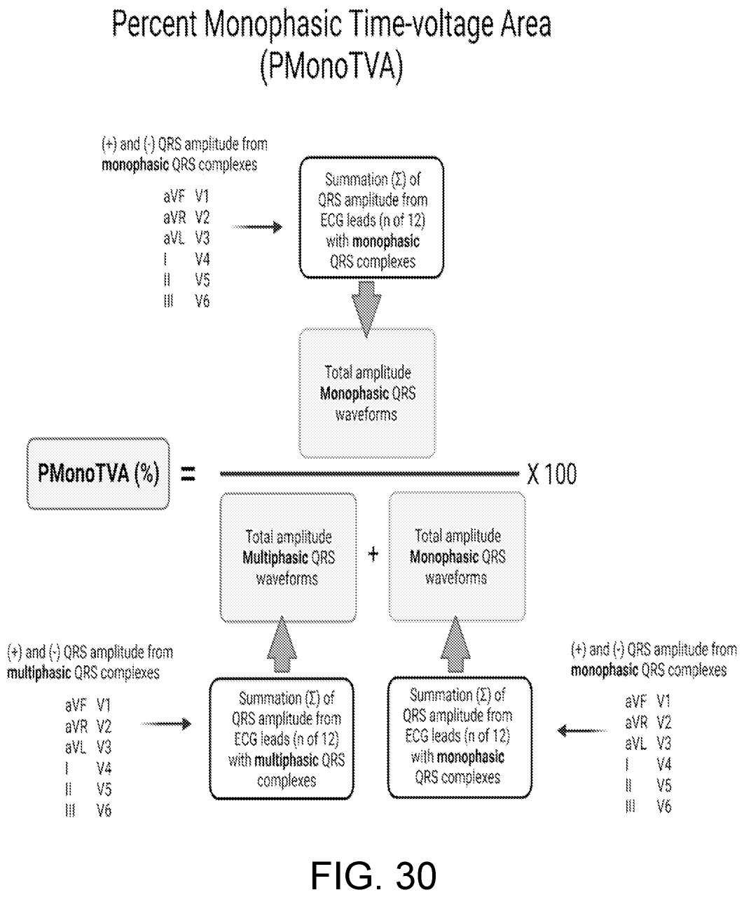

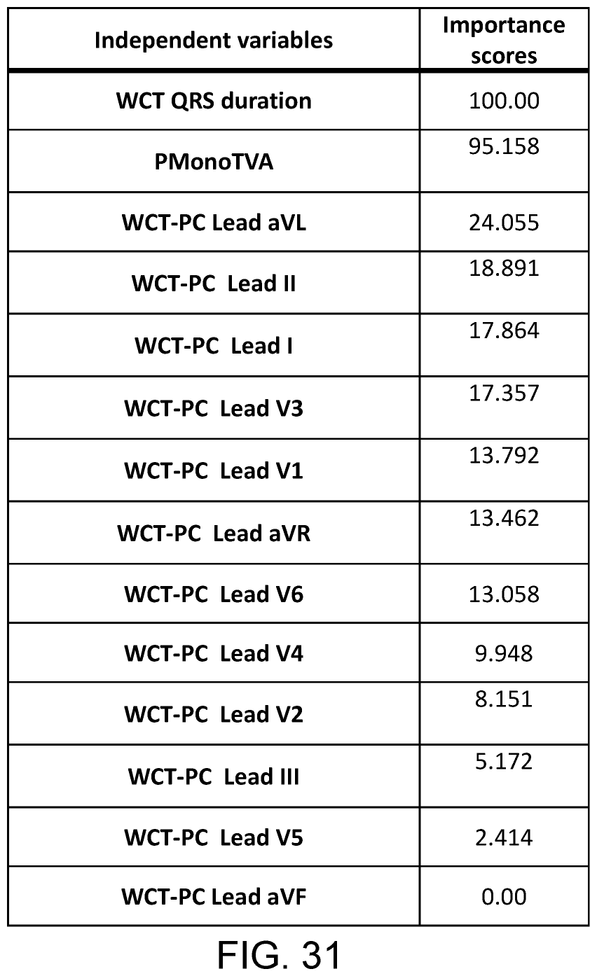

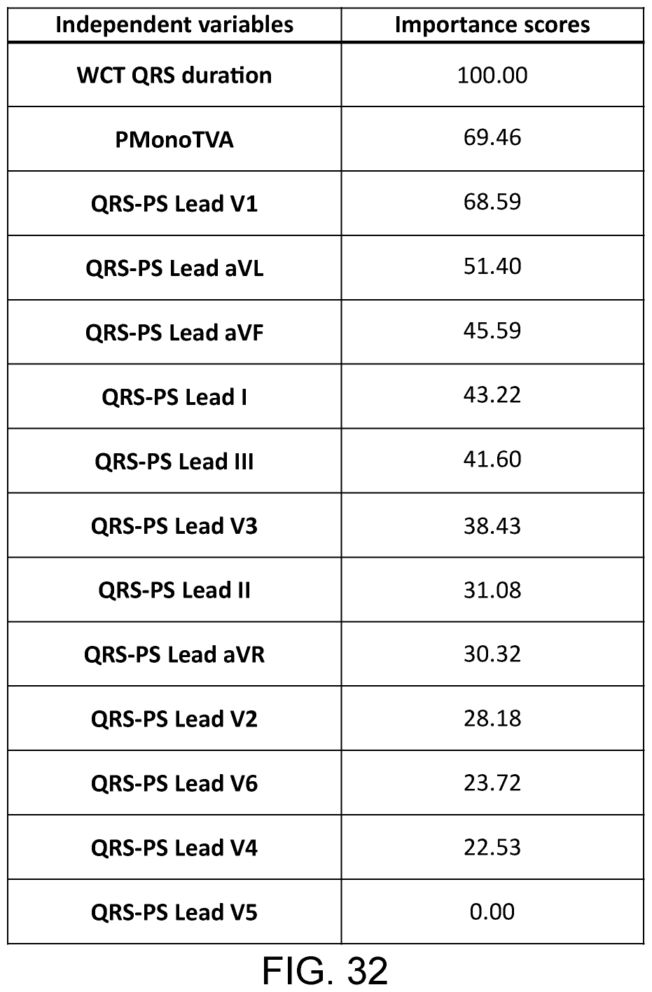

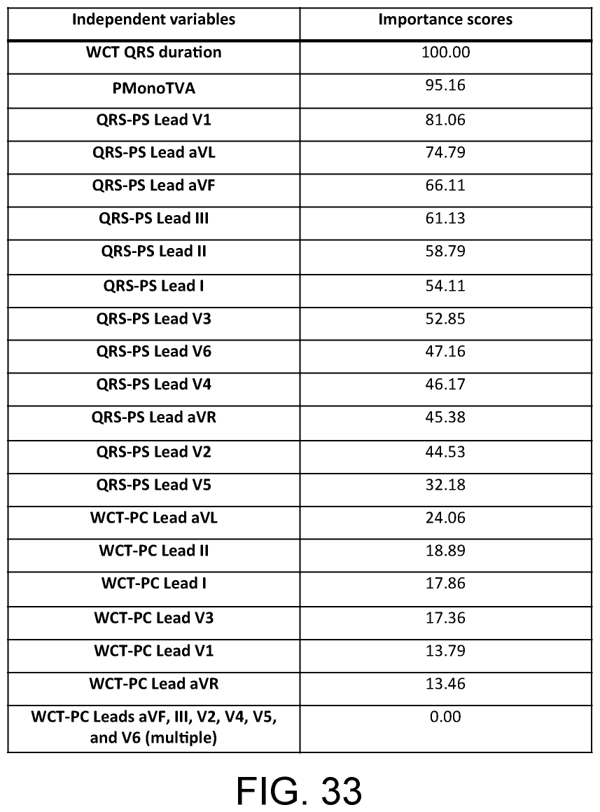

FIG. 30 illustrates an exemplary schematic flow chart of the percent monophasic time-voltage area calculation. PMonoTVA calculation is derived from measured QRS waveform TVA (time-voltage areas) (μV·ms) of the dominant QRS complex template within each lead of the 12-lead ECG. FIG. 31 illustrates an exemplary table of importance scores for RF Models in Part 1. FIG. 32 illustrates an exemplary table of importance scores for RF Models in Part 2. FIG. 33 illustrates an exemplary table of importance scores for RF Models in Part 3.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

Wide QRS complex tachycardia (WCT) differentiation into ventricular tachycardia (VT) and supraventricular wide complex tachycardia (SWCT) remains clinically challenging despite numerous manual 12-lead electrocardiogram (ECG) criteria and algorithms. More recently, automated solutions, which leverage computerized electrocardiogram interpretation (CEI) software measurements, offer an effective and practical means to accurately differentiate WCTs.

Automated WCT differentiation algorithms are proposed that make use of (i) WCT QRS complex polarity direction (i.e., WCT Polarity Code (WCT-PC)) and (ii) QRS polarity changes between the WCT and baseline ECG (i.e., QRS Polarity Shift (QRS-PS)).

In a three-part investigation machine learning (ML) models (logistic regression (LR), artificial neural network (ANN), Random Forests (RF), support vector machine (SVM)), and ensemble learning (EL)) comprised of WCT-PC and QRS-PS, as well as previously reported WCT differentiation features were derived and validated. In Part 1, ML models were trained and tested using features derived from measurements of the WCT ECG alone. In Part 2, ML models were trained and tested using features derived from measurements of paired WCT and baseline ECGs. In Part 3, ML models that incorporate all features used in Part 1 and 2 were trained and tested.

Of the 235 consecutive patients presenting with WCT in the testing cohort, 103 heart rhythm diagnoses (i.e., VT or SWCT) were established by a diagnostic gold standard. Among 103 WCT patients (48 VT and 55 SWCT) with a gold standard diagnosis, ML models in Part 1 achieved similar overall diagnostic performance, with an AUC range of 0.86 to 0.91. In Part 2, among patients with a gold standard diagnosis, ML models displayed similar performance, with AUC values ranging from 0.90 to 0.94. In Part 3, most ML models demonstrated comparable diagnostic performance among patients with a gold standard diagnosis, with AUC values ranging from 0.72 to 0.93.

Accurate differentiation of WCTs can be achieved by integrating features like WCT-PC and QRS-PS into ML models.

In order to counter the practical diagnostic limitations of manual algorithms, as well as supplement their known strengths, automated WCT differentiation algorithms were developed and validated using computerized ECG interpretation (CEI) software. The core aspect of automated approaches lies in the utilization of readily available computerized ECG measurements (such as WCT QRS duration) and innovative customized features (like horizontal and frontal percent amplitude change (PAC)) for the differentiation of VT and SWCT through machine learning (ML) techniques. In this context, WCT differentiation algorithms are also introduced that harness new innovative features and ML modeling techniques. Specifically ML models are introduced that utilize features describing the polarity of the WCT QRS complex (referred to as WCT Polarity Code (WCT-PC)) and polarity shifts in the QRS complex between the WCT and the baseline ECG (referred to as QRS Polarity Shift (QRS-PS)) among leads comprising the standard 12-lead ECG.

In a three-part investigation, WCT differentiation models comprised of features derived from WCT and baseline ECG data were developed, trialed, and compared. In Part 1, different machine learning (ML) models were trained and tested using features derived from computerized ECG measurements present on the WCT ECG alone. Herein (in Part 1), features relating to the orientation of QRS complex polarity in all ECG leads during the WCT itself (i.e., WCT-PC) are evaluated. In Part 2, ML models were trained and tested using features that may be formulated from paired WCT and baseline ECGs. Herein (in Part 2), features relating to the presence or absence of QRS polarity shifts between the WCT and baseline ECG (i.e., QRS-PS) are evaluated. In Part 3, we trained and tested ML models which incorporate all features used in Part 1 and 2 were trained and tested. The overarching structure and methodology of the study are visually detailed in FIGS. 11A-11C.

All paired WCT and baseline ECGs were recorded within clinical settings. ECGs were standard 12-lead recordings (paper speed: 25 mm/s and voltage calibration: 10 mm/mV) accessed from data archives provided by an ECG interpretation software system. WCTs were required to satisfy standard WCT criteria (QRS duration ≥120 ms and ventricular rate ≥100 beats per minute) and possess an official ECG interpretation of (i) ‘ventricular tachycardia’, (ii) ‘supraventricular tachycardia’, or (iii) ‘wide complex tachycardia’. Baseline ECGs were defined as the first non-WCT rhythm recorded after the WCT event.

Polymorphic WCTs and WCTs demonstrating grossly irregular atrioventricular conduction (e.g., atrial fibrillation or atrial flutter with variable atrioventricular block) were excluded, and ECGs demonstrating truncated WCTs (e.g., a brief run of non-sustained VT) occurring within a dominant baseline heart rhythm (e.g., normal sinus rhythm) were not evaluated. If a WCT did not have a baseline ECG or definitive clinical diagnosis established by the patient's overseeing physician, it was excluded from further analysis. Among patients having multiple WCT events, the surplus of WCT ECGs occurring after the first WCT event were excluded (i.e., one WCT and baseline ECG pair was evaluated per patient).

Heart rhythm diagnoses (i.e., VT or SWCT) were established by the patient's supervising physician. Physicians responsible for clinical diagnoses were stratified according to a hierarchy of clinical expertise: (i) heart rhythm cardiologist, (ii) non-heart rhythm cardiologist, and (iii) non-cardiologist. Heart rhythm diagnoses were organized according to whether they were supported by a corroborating EP or implantable intracardiac device recordings (i.e., gold standard cohort vs. non-gold standard cohort).

For the first institution, the training cohort comprised 421 consecutive patients with paired WCT and baseline ECGs acquired at the Mayo Clinic Rochester or Mayo Clinic Health System of Southeastern Minnesota (Sep. 1, 2011, through Nov. 30, 2016). Of the 421 patients, 192 heart rhythm diagnoses (i.e., VT or SWCT) were established with a corroborating electrophysiological procedure (EP) or implantable intracardiac device recordings (i.e., gold standard cohort). Of the 421 patients, 229 heart rhythm diagnoses were established without a corroborating EP or implantable intracardiac device recordings. The ECG selection processes and clinical characteristics of this patient cohort are thoroughly described in previous reports. The Mayo Clinic Institutional Review Board approved patient data acquisition and analysis.

For the second institution, the testing cohort comprised 235 consecutive patients with paired WCT and baseline ECGs obtained at Barnes-Jewish Hospital in St. Louis (Jan. 1, 2012, through Dec. 31, 2014). FIG. 28 shows a flow diagram of testing cohort selection. Of the 235 patients, 103 heart rhythm diagnoses (i.e., VT or SWCT) were established with a corroborating EP or implantable intracardiac device recordings (i.e., gold standard cohort). Of the 235 patients, 132 heart rhythm diagnoses were established without a corroborating EP or implantable intracardiac device recordings (i.e., non-gold standard cohort). Patient data acquisition and analysis was approved by the Human Research Protection Office of Washington University in St. Louis, Missouri.

Standard computerized ECG measurements for WCT and baseline ECGs were automatically generated by the ECG interpretation software. Computerized QRS amplitude (μV) and TVA (time-voltage area [μV·ms]) measurements of waveforms above (r/R and r′/R′) and below (q/QS, s/S, and s′/S′) the isoelectric baseline were automatically derived from the dominant QRS complex template within each lead of the 12-lead ECG (FIG. 29). Only amplitude and TVA measurements representative of QRS complex waveforms were analyzed. Measurements of the ventricular pacing stimuli or ECG artifact were excluded from our analysis.

The WCT differentiation features includes:

WCT QRS duration (ms)—QRS duration of the WCT was automatically generated by the ECG interpretation software package.

Percent monophasic time-voltage area (%)—PMonoTVA is the percentage (%) of QRS TVA contained by monophasic QRS complexes on the 12-lead ECG. This parameter is calculated as the percentage ratio of the sum of QRS TVA from ECG leads with monophasic QRS complexes (i.e., monophasic TVA) to the entire sum of QRS TVA from all ECG leads (i.e., monophasic TVA plus multiphasic TVA) (FIG. 30). Representative TVA measurements are attained from QRS complex waveforms (q/QS, r/R, s/S, r′/R′, and s′/S′) of the dominant QRS complex template of individual leads of the 12-lead ECG.

WCT Polarity Code (WCT-PC)—The term WCT-PC denotes the direction of QRS polarity (i.e., positive, negative, or equiphasic) for individual leads of the recorded WCT. Specifically, QRS complex polarity refers to the dominant direction (i.e., upright, downward, or equiphasic) to which individual QRS complexes of the 12-lead ECG are oriented (FIG. 12).

For this analysis, positive, negative, and equiphasic QRS polarity was determined by the sum (Σ) of QRS complex waveform amplitudes (μV) above (r/R and r′/R′) and below (q/QS, s/S, and s′/S′) the isoelectric baseline. For calculation purposes, the amplitude (μV) of QRS complex waveforms having a downward orientation (i.e., q/QS, s/S, and s′/S′) were considered numerically negative, while the amplitude (μV) of QRS complex waveforms having an upright orientation (i.e., r/R and r′/R′) were considered numerically positive. If the sum of QRS complex waveforms (i.e., q/QS+r/R+s/S+r′/R′+s′/S′) was greater than 250 μV (i.e., 2.5 grid boxes) above the isoelectric baseline (i.e., Σ QRS amplitude >250 μV), the QRS complex is defined as having positive polarity. If the sum of QRS complex waveforms was more than 250 μV beneath the isoelectric baseline (i.e., Σ QRS amplitude <−250 μV), the QRS complex is defined as having negative polarity. If the sum of QRS complex waveforms falls between 250 μV above or beneath the isoelectric baseline (i.e., 250 μV<=Σ QRS amplitude=>−250 μV), the QRS complex is defined as having equiphasic polarity.

Alternatively, QRS polarity may be determined using QRS TVAs. As illustrated in FIG. 29, positive (+), negative (−), and equiphasic (=) QRS polarity was determined by the sum (Σ) of QRS complex waveform TVAs (μV·ms) above (r/R and r′/R′) and below (q/QS, s/S, and s′/S′) the isoelectric baseline. For calculation purposes, the TVA of QRS complex waveforms having a downward orientation (i.e., q/QS, s/S, and s′/S′) were considered numerically negative, while the TVA of QRS complex waveforms having an upright orientation (i.e., r/R and r′/R′) were considered numerically positive. If the sum of QRS complex waveforms (i.e., q/QS+r/R+s/S+r′/R′+s′/S′) was greater than 250 μV·ms above the isoelectric baseline (i.e., Σ QRS TVA >250 μV·ms), the QRS complex is defined as having positive (+) polarity. If the sum of QRS complex waveforms was more than 250 μV·ms beneath the isoelectric baseline (i.e., Σ QRS TVA <−250 μV·ms), the QRS complex is defined as having negative (−) polarity. If the sum of QRS complex waveforms falls between 250 μV·ms above or beneath the isoelectric baseline (i.e., 250 μV·ms<=>Σ QRS TVA=>−250 μV ms), the QRS complex is defined as having equiphasic (=) polarity.

QRS Polarity Shift (QRS-PS)—The term QRS-PS will be used to describe changes that may or may not occur in QRS polarity between a baseline and WCT rhythm for individual ECG leads. Characterization of QRS polarity changes, or lack thereof, can be organized according to the type of change between a baseline and WCT rhythm (FIGS. 13 and 14). In specific terms, polarity changes between a WCT and baseline ECG may be characterized in one of 9 ways (WCT ECG→Baseline ECG or vice versa): (i) equiphasic (=)→equiphasic (=), (ii) positive (+)→positive (+), (iii) negative (−)→negative (−), (iv) positive (+)→negative (−), (v) positive (+)→equiphasic (=), (vi) negative (−)→positive (+), (vii) negative (−)→equiphasic (=), (viii) equiphasic (=)→positive (+), and (ix) equiphasic (=)→negative (−). In general terms, polarity changes between a WCT and baseline ECG may be characterized in one of 3 ways: (i) polarity shift, (ii) partial polarity shift, or (iii) no polarity shift.

For this analysis, polarity shift is defined by the occurrence of a change (or shift) in QRS complex polarity between the WCT and baseline ECG. For a polarity shift to occur, individual QRS complexes of the 12-lead ECG having a dominate QRS polarity (positive or negative) on the baseline or WCT ECG must transform into the opposite dominate QRS polarity on the corresponding WCT or baseline ECG, respectively. For example, a polarity shift has occurred if an individual ECG lead demonstrates positive QRS complex polarity during the baseline heart rhythm that later transforms into a negative QRS complex polarity during a WCT. Similarly, a polarity shift is also present when an ECG lead demonstrates negative QRS complex polarity during the baseline heart rhythm but a positive QRS complex polarity during a WCT. Partial polarity shift is defined by QRS morphology transformation into or out of an equiphasic QRS complex. For a partial polarity shift to occur, individual QRS complexes of baseline or WCT ECG must either transform into or out of an equiphasic QRS complexes on the corresponding baseline or WCT ECG. No polarity shift is defined by the absence of QRS polarity changes for individual leads between the baseline and WCT ECGs. For example, no polarity shift is present when an individual ECG lead demonstrates positive QRS complex polarity during the baseline heart rhythm and WCT. Similarly, no polarity shift is present for an individual ECG lead demonstrating a negative QRS complex or equiphasic polarity for both the baseline and WCT ECGs. FIG. 15 offers examples of polarity shift, partial polarity shift, and no polarity shift for ECG leads of the frontal and horizontal plane.

In Part 1, five ML modeling techniques (logistic regression (LR), artificial neural network (ANN), Random Forests (RF), support vector machine (SVM), and ensemble learning (EL)) were used to train and test binary classification models that may be implemented on the WCT ECG alone, with each model incorporating fourteen covariates: WCT QRS duration (ms), PMonoTVA (%), and WCT-PCECG lead X (X12 covariates in total [one per ECG lead]). For any given ECG lead, WCT-PC had three potential categorical values: (i) positive, (ii) negative, or (iii) equiphasic.

In Part 2, the five ML modeling techniques were used to train and test binary classification models that may be implemented on paired WCT and baseline ECGs, with each model incorporating fourteen covariates: WCT QRS duration (ms), PMonoTVA (%), and QRS-PSECG lead X (X12 covariates in total [one per ECG lead]. For any given ECG lead, QRS-PS covariates had three potential categorical options: (i) polarity shift, (ii) partial polarity shift, or (iii) no polarity shift.

In Part 3, the five ML modeling techniques were used to train and test binary classification models incorporating twenty-six covariates: WCT QRS duration (ms), PMonoTVA (%), WCT-PCECG lead X (X12 covariates in total [one per ECG lead]), and QRS-PSECG lead X (X12 covariates in total [one per ECG lead]). For any given ECG lead, WCT-PC had three potential categorical values: (i) positive, (ii) negative, or (iii) equiphasic. Similarly, for any given ECG lead, QRS-PS covariates had 3 potential categorical options: (i) polarity shift, (ii) partial polarity shift, or (iii) no polarity shift.

Categorical variables were compared using Chi-square tests. Wilcoxon rank-sum tests were used to compare continuous variables. Positive likelihood ratios (+LR) were used to evaluate the individual discriminatory capacity of WCT-PCECG lead X and QRS-PSECG lead X for the correct rhythm diagnosis. Outlier values for each parameter were winsorized to diminish undue influence on model coefficients. Heart rhythm classification (i.e., VT or SWCT) by each model was established using a pre-specified VT probability partition of 50% (i.e., VT≥50% and SWCT<50%). Performance metrics (i.e., accuracy, sensitivity, specificity, and AUC) for each model were assessed according to their agreement with the correct heart rhythm diagnosis. A comparison of fit between ML models was completed using a Delong test. All statistical analysis was performed using R Statistical Software (R Foundation for Statistical Computing, Vienna, Austria).

Clinical characteristics of the training cohort for Institute 1 are shown in FIG. 6. Among training cohort patients, the VT group included more patients with coronary artery disease, prior myocardial infarction, ongoing antiarrhythmic drug use, ischemic cardiomyopathy, and an implantable cardioverter-defibrillator (ICD). The SWCT group included more patients with an implanted pacemaker. The VT group comprised more patients with a severely depressed (≤30%) left ventricular ejection fraction (LVEF), whereas the SWCT group included more patients with a preserved (≥50%) LVEF. A majority of patients with VT diagnoses had a corroborating EP or intracardiac device recording. Conversely, a minority of SWCT patients had a corroborating EP or intracardiac device recording.

Clinical characteristics of the testing cohort for Institution 2 are shown in FIG. 7. Among testing cohort patients, the VT group included more patients with ongoing antiarrhythmic drug use, ischemic cardiomyopathy, and an ICD. Similar to the training cohort, the VT group comprised more patients with a severely depressed (≤30%) LVEF, while the SWCT group included more patients with a preserved (≥50%) LVEF. Similar to the training cohort, most VT patients, and a minority of SWCT patients, had a corroborating EP or intracardiac device recording.

The frequency of various types of WCT Polarity Codes (WCT-PCs) for VT and SWCT groups within the testing cohort is shown in FIG. 19. The discriminatory capacity of positive and negative QRS polarity among individual ECG leads of the WCT is illustrated in FIG. 14. For the frontal ECG plane, positive QRS polarity in lead aVR modestly favored VT in both the training and testing cohorts. Positive QRS polarity in lead aVF and III appeared to marginally favor VT in the training cohort only. Negative QRS polarity complexes in leads aVL and I modestly favored VT in both the training and testing cohorts. Conversely, a negative QRS polarity in lead III slightly favored SWCT. Otherwise, QRS complex polarity patterns in the frontal ECG plane did not significantly favor VT or SWCT. For the horizontal ECG plane, positive QRS polarity in V4 and V5 marginally favored VT in the training and testing cohorts. Positive QRS polarity in leads V2 and V3 slightly favored VT in the training cohort only; but in lead V1, positive QRS polarity slightly favored VT in the testing cohort. Negative QRS polarity complexes slightly favored SWCT in leads V3 and V4 in the training cohort only. Otherwise, QRS complex polarity patterns in the horizontal ECG plane did not significantly favor VT or SWCT.

The frequency of various types of QRS Polarity Shifts (QRS-PSs) for VT and SWCT groups within the testing cohort is shown in FIGS. 20A, 20B, and 21A-21D. The discriminatory capacity of any polarity shift occurring among leads of the 12-lead ECG is illustrated in FIG. 15. For all ECG leads the presence of a polarity shift strongly favored VT (as opposed to SWCT) for both the training and testing cohorts. Conversely, for all ECG leads the absence of a polarity shift strongly favored SWCT (as opposed to VT) in both the training and testing cohorts.

### Part 1: ML Models—WCT ECG Data Alone

Diagnostic performance metrics of the five ML models in the training cohort are summarized in FIG. 22. FIG. 8 demonstrates the diagnostic performance of various modeling subtypes as applied to subjects with a gold standard and non-gold standard diagnosis in the testing cohort, respectively. In the gold standard cohort, ML model techniques demonstrated similar diagnostic performance (AUC range: 0.86 to 0.91). In the non-gold standard cohort, ML model techniques demonstrated similar diagnostic performance (AUC range: 0.85 to 0.96). Specific tuning parameters used for ML model training are shown in FIG. 24. The importance of various WCT differentiation features of the RF model is shown in FIG. 31.

### Part 2: ML Models—Paired WCT and Baseline ECG Data

Diagnostic performance metrics of the five ML models, when applied to the training cohort, are summarized in FIG. 25. FIG. 9 demonstrates the diagnostic performance of the ML modeling subtypes when applied to subjects with a gold standard and non-gold standard diagnosis in the testing cohort, respectively. Among subjects with a gold standard diagnosis, ML model techniques demonstrated similar diagnostic performance (AUC range: 0.90 to 0.94). Among subjects with a non-gold standard diagnosis, ML model techniques demonstrated similar diagnostic performance (AUC range: 0.89 to 0.95). Specific tuning parameters used for ML model training are shown in FIG. 25. The importance of various WCT differentiation features of the RF model is shown in FIG. 32.

### Part 3: ML Models—WCT ECG Data+Paired WCT and Baseline ECG Data

Diagnostic performance metrics of the five ML models, when applied to the training cohort, are summarized in FIG. 26. FIG. 10 demonstrates the diagnostic performance of the ML modeling subtypes when applied to subjects with a gold standard and non-gold standard diagnosis in the testing cohort, respectively. In the gold standard cohort, ML models demonstrated varying capabilities in diagnostic performance with AUC values ranging from 0.72 to 0.93. Among subjects with a non-gold standard diagnosis, ML model techniques demonstrated varying diagnostic performance with AUC values ranging from 0.79 to 0.93. Specific tuning parameters used for ML model training are shown in FIG. 27. The importance of various WCT differentiation features of the RF model is shown in FIG. 33. In the RF model, QRS-PSECG lead X variables possessed higher importance ratings than WCT-PCECG lead X variables.

In this work, diagnostic features (i.e., WCT-PCs and QRS-PS) able to be incorporated into automated ML models designed to perform accurate WCT differentiation were described and analyzed. Models were trained and tested using computerized ECG data from (i) WCT ECG alone (Part 1) and (ii) paired WCT and baseline ECGs (Parts 2 and 3). In Part 1, it was observed that the incorporation of features derived from the WCT data alone (e.g., WCT-PCs) into various ML modeling techniques resulted in favorable diagnostic performance. In Parts 2 and 3, it was found that features derived from paired WCT and baseline ECG data (i.e., QRS-PS) resulted in improved diagnostic performance. However, the aggregation of all available features (used in Part 1 and 2) in Part 3 did not lead to a meaningful difference in diagnostic performance compared to Part 2. Lastly, it was observed that ML model performance was maintained irrespective of its implementation on patients who have or don't have a corroborating gold standard diagnosis.

It was investigated whether WCT Polarity Codes (WCT-PCs) (i.e., positive, negative, or equiphasic) of individual leads of the WCT itself can be leveraged to arrive at an accurate VT or SWCT diagnosis. It was hypothesized that VT and SWCT rhythms would in many cases demonstrate a unique ‘WCT-PC signature’ that would enable accurate WCT differentiation. For example, WCT rhythms with a ‘northwest axis’ ([+] QRS polarity in lead aVR) and a ‘QS pattern’ in lead V6 ([−] QRS polarity in V6) are more likely to be VT, especially since this WCT-PC signature would not be expected among patients SWCT due to aberrancy. Similarly, one would expect WCT rhythms with a rightward axis ([−] QRS polarity in lead I) and left bundle branch block (LBBB) pattern ([−] QRS polarity in lead V1) would be more consistent with VT than SWCT. Prior authors have identified both aforementioned examples as patterns as being more consistent with VT. Therefore it was sought to determine whether WCT-PC signatures (known or unknown) could be used to differentiate VT and SWCT. As such, WCT-PCs were incorporated into various ML modeling techniques and methods capable of assimilating features with disparate non-parametric relationships for the purpose of VT and SWCT classification.

After assigning positive, negative, or equiphasic labels for all QRS complexes for individual leads of the recorded WCT, relationships were detected between QRS complex polarity and the underlying WCT diagnosis. For example, it was observed that a positive QRS polarity in lead aVR favored VT in both the training and testing cohorts. Similarly, it was observed that a negative QRS polarity in lead I favored VT in both the training and testing cohorts. Nonetheless, among the few ECG leads that did demonstrate a relationship between QRS complex polarity and the underlying WCT diagnosis, the influence of WCT-PC was objectively small. Yet, upon incorporating WCT-PCs of all leads into various ML modeling techniques, it was observed that WCT-PCs can help differentiate WCTs. ML methods appeared to decipher complex non-parametric relationships between WCT-PCs to achieve a strong overall diagnostic performance.

It was evaluated whether the broad characterization of QRS polarity shifts (QRS-PS) between the WCT and baseline ECG could be leveraged in discriminating VT and SWCT. It was hypothesized that the presence of any polarity shift (positive QRS polarity transforming into negative QRS polarity, or vice versa) between the baseline and WCT ECGs would be highly predictive of VT. Conversely, it was expected that the absence of a polarity shift would be more consistent with SWCT. The electrophysiological basis for the hypotheses relates to the differences by which VT and SWCT ordinarily depolarize the ventricular myocardium. In the case of SWCT, it is quite common for the means of ventricular depolarization to be the same or very similar to that of the baseline heart rhythm (e.g., normal sinus rhythm). As such, it is unusual for a polarity shift to occur among SWCTs; rather, SWCTs would more commonly demonstrate a lack of any polarity shift. On the other hand, VT is known to demonstrate substantial ‘electrical freedom’ compared to its relatively constrained SWCT counterpart. Considering that VT can originate and spread from any part of the left or right ventricles, it can produce ventricular depolarization wavefronts that move in the opposite direction of the baseline heart rhythm, essentially demonstrating a complete reversal of the mean electrical vector orientation. As such, many VT rhythms would be expected to elicit a polarity shift between the baseline and WCT rhythm. This concept was similarly leveraged by other recently described features, (e.g., frontal and horizontal PAC) that were incorporated into WCT differentiation algorithms.

In this analysis, it was observed that any polarity shift is highly predictive of VT. Conversely, it was observed that the absence of polarity shift is highly predictive of SWCT. Upon incorporating QRS-PS covariates from all twelve ECG leads into various ML modeling techniques, it was observed a robust capability for accurately differentiating VT and SWCT. In both Parts 2 and 3 of this analysis ML models yielded strong performance using QRS-PS covariates. Moreover, it was observed that QRS-PS covariates have uniformly stronger influence than WCT-PC covariates, as demonstrated by RF importance scores.

Ideally, accurate and reliable discrimination of VT and SWCT would occur automatically upon 12-lead ECG acquisition. Unfortunately, CEI (computerized ECG interpretation) software programs have not yet achieved sufficient diagnostic accuracy for the interpretation of many complex heart rhythms, including WCTs. At present, contemporary CEI software packages do not reliably differentiate WCTs; rather, instead, most recorded WCT events are given a generic label of ‘wide complex tachycardia’, which offers little-to-no assistance to clinicians. Consequently, clinicians faced with patients exhibiting WCT must rely on conventional manual ECG interpretation techniques to accurately and promptly diagnose VT or SWCT. To do this effectively, clinicians should meticulously analyze a well-recorded 12-lead ECG that accurately represents the WCT event and diligently apply the specific electrocardiogramaria outlined by traditional interpretation methods. Unfortunately, this procedure is commonly thwarted by the improper application or lack of use of manual WCT differentiation methods. As such, the application of manual ECG interpretation algorithms or criteria is unsurprisingly problematic-especially for non-expert clinicians who must promptly diagnose and manage high-acuity patients.

In this work, means to distinguish VT and SWCT accurately using ECG data provided by the (i) WCT alone and (ii) paired WCT and baseline ECGs were developed and trialed. Similar to other recently described methods, it was again demonstrated how automated approaches to differentiate WCTs may be developed through the use of available data provided by CEI software. Likewise, readily available ECG data were transformed into mathematically formulated features (e.g., QRS-PS), which may in turn be incorporated into models that provide clinicians an impartial binary classification or estimation of VT likelihood (i.e., 0.00% to 99.99% VT probability).

A recognized limitation of recently described automated WCT differentiation methods is that they require computerized data provided by both the WCT ECG and its corresponding baseline ECG. In Part 1 of this analysis means to distinguish VT and SWCT using the WCT alone were described. Thus irrespective of the presence of a baseline ECG, ML models may be employed. For instance, if a patient does not have a baseline ECG, ML models that use computerized ECG data of the WCT alone can be used. However, if a patient already possesses a digitally archived baseline ECG, or if a new baseline ECG is recorded after the WCT event, VT or SWCT classification may be executed by ML models that leverage paired WCT and baseline ECG comparisons, and thereby supersede the classification from ML models that only use computerized ECG data provided by the WCT itself. FIGS. 16A and 16B demonstrates a proposed framework for the application of WCT differentiation algorithms according to the presence or absence of a baseline ECG.

Ideally, healthcare professionals should be able to combine automatically generated VT probabilities, produced by automated algorithms, with diagnoses derived from traditional WCT differentiation methods, such as the Brugada algorithm. In a recent analysis, it was observed that displaying VT probability (or likelihood that the WCT is VT) generated by the VT Prediction Model helped physician's diagnostic performance in discriminating VT and SWCT. By similar means, commercially available ECG interpretation software platforms that incorporate ML models would be able to help clinicians discriminate VT and SWCT accurately.

Automated ML algorithms are presented that leverage features relating to WCT QRS complex polarity (i.e., WCT-PC) and QRS complex polarity shifts between the WCT and baseline ECG (i.e., QRS-PS). By these means, accurate VT and SWCT classification may be accomplished using readily available CEI data provided by the (i) WCT alone and (ii) paired WCT and baseline ECGs.

### Machine Learning & Other Matters

The computer-implemented methods discussed herein may include additional, less, or alternate actions, including those discussed elsewhere herein. The methods may be implemented via one or more local or remote processors, transceivers, and/or sensors (such as processors, transceivers, and/or sensors mounted on vehicles or mobile devices, or associated with smart infrastructure or remote servers), and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as image, mobile device, vehicle telematics, and/or intelligent home telematics data. The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing-either individually or in combination. The machine learning programs may also include natural language processing, semantic analysis, automatic reasoning, and/or machine learning.

In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In one embodiment, machine learning techniques may be used to extract the relevant personal belonging and/or home feature information for customers from health sensors, patient monitoring devices, mobile device sensors, vehicle-mounted sensors, home-mounted sensors, and/or other sensor data, image data, and/or other data.

### Additional Considerations

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps,” or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

As used herein, the term “database” can refer to either a body of data, a relational database management system (RDBMS), or to both. As used herein, a database can include any collection of data including hierarchical databases, relational databases, flat file databases, object-relational databases, object-oriented databases, and any other structured collection of records or data that is stored in a computer system. The above examples are example only, and thus are not intended to limit in any way the definition and/or meaning of the term database. Examples of RDBMS' include, but are not limited to including, Oracle® Database, MySQL, IBM® DB2, Microsoft® SQL Server, Sybase®, and PostgreSQL. However, any database can be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In another example, a computer program is embodied on a computer-readable medium. In an example, the system is executed on a single computer system, without requiring a connection to a server computer. In a further example, the system is being run in a Windows® environment (Windows is a registered trademark of Microsoft Corporation, Redmond, Washington). In yet another example, the system is run on a mainframe environment and a UNIX® server environment (UNIX is a registered trademark of X/Open Company Limited located in Reading, Berkshire, United Kingdom). In a further example, the system is run on an iOS® environment (iOS is a registered trademark of Cisco Systems, Inc. located in San Jose, CA). In yet a further example, the system is run on a Mac OS® environment (Mac OS is a registered trademark of Apple Inc. located in Cupertino, CA). In still yet a further example, the system is run on Android® OS (Android is a registered trademark of Google, Inc. of Mountain View, CA). In another example, the system is run on Linux® OS (Linux is a registered trademark of Linus Torvalds of Boston, MA). The application is flexible and designed to run in various different environments without compromising any major functionality.

As used herein, an element or step recited in the singular and proceeded with the word “a” or “an” should be understood as not excluding plural elements or steps, unless such exclusion is explicitly recited. Furthermore, references to “example” or “one example” of the present disclosure are not intended to be interpreted as excluding the existence of additional examples that also incorporate the recited features. Further, to the extent that terms “includes,” “including,” “has,” “contains,” and variants thereof are used herein, such terms are intended to be inclusive in a manner similar to the term “comprises” as an open transition word without precluding any additional or other elements.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

Furthermore, as used herein, the term “real-time” refers to at least one of the time of occurrence of the associated events, the time of measurement and collection of predetermined data, the time to process the data, and the time of a system response to the events and the environment. In the examples described herein, these activities and events occur substantially instantaneously.

In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

The computer-implemented methods discussed herein can include additional, less, or alternate actions, including those discussed elsewhere herein. The methods can be implemented via one or more local or remote processors, transceivers, servers, and/or sensors (such as processors, transceivers, servers, and/or sensors mounted on vehicles or mobile devices, or associated with smart infrastructure or remote servers), and/or via computer-executable instructions stored on non-transitory computer-readable media or medium. Additionally, the computer systems discussed herein can include additional, less, or alternate functionality, including that discussed elsewhere herein. The computer systems discussed herein can include or be implemented via computer-executable instructions stored on non-transitory computer-readable media or medium.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein can be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer device for classifying a wide complex tachycardia (WCT) pattern of a subject, the computer device comprising at least one processor in communication with at least one memory device, wherein the at least one processor is programmed to:
receive WCT electrocardiogram (ECG) data indicative of a WCT pattern;
transform the WCT ECG data into at least one engineering feature;
execute at least one machine learning model to analyze the at least one engineering feature and to output a classification of the WCT pattern;
based upon the classification of the WCT pattern, determine whether the WCT pattern is indicative of at least one of a ventricular tachycardia (VT) and a supraventricular wide complex tachycardia (SWCT); and
select a treatment for the subject based upon the determination.

2. The computer device of claim 1, wherein the at least one processor is further programmed to receive the WCT ECG data from a 12 lead ECG device.

3. The computer device of claim 1, wherein the at least one processor is further programmed to determine whether the WCT pattern is indicative of at least one of a ventricular tachycardia, a supraventricular wide complex tachycardia, a probability of a VT, and a probability of an SWCT.

4. The computer device of claim 1, wherein the at least one engineered feature includes at least one of WCT QRS duration (ms), PMonoTVA (%), WCT Polarity Code (WCT-PC) for each lead of the ECG data, and any combination thereof.

5. The computer device of claim 4, wherein the at least one processor is further programmed to select at least one engineering feature for the transformation.

6. The computer device of claim 1, wherein each WCT-PC for each lead of the ECG data is selected from positive, negative, or equiphasic.

7. The computer device of claim 1, wherein the machine learning model is selected from a logistic regression (LR) model, an artificial neural network (ANN), a Random Forests (RF) model, a support vector machine (SVM), and an ensemble learning (EL) model.

8. A computer device for classifying a wide complex tachycardia (WCT) pattern of a subject, the computer device comprising at least one processor in communication with at least one memory device, wherein at least one processor is programmed to:
receive WCT ECG data indicative of the WCT pattern and baseline ECG data;
transform the WCT ECG data and baseline ECG data into at least one engineered feature;
execute at least one machine learning model to analyze the at least one engineering feature and to output a classification of the WCT pattern;
based upon the classification of the WCT pattern, determine whether the WCT pattern is indicative of at least one of a ventricular tachycardia (VT) and a supraventricular wide complex tachycardia (SWCT); and
select a treatment for the subject based upon the determination.

9. The computer device of claim 8, wherein the at least one processor is further programmed to receive the WCT ECG data from a 12 lead ECG device.

10. The computer device of claim 8, wherein the at least one processor is further programmed to determine whether the WCT pattern is indicative of at least one of a ventricular tachycardia, a supraventricular wide complex tachycardia, a probability of a VT, and a probability of an SWCT.

11. The computer device of claim 8, wherein the at least one engineered feature includes at least one of WCT QRS duration (ms), PMonoTVA (%), QRS-PS for each lead of the ECG data, and any combination thereof.

12. The computer device of claim 11, wherein the at least one processor is further programmed to select at least one engineering feature for the transformation.

13. The computer device of claim 11, wherein each QRS-PS for each lead of the ECG data is selected from equiphasic (=)→equiphasic (=), positive (+)→positive (+), negative (−)→negative (−), positive (+)→negative (−), positive (+)→equiphasic (=), negative (−)→positive (+), negative (−)→equiphasic (=), equiphasic (=)→positive (+), or equiphasic (=)→negative (−).

14. The computer device of claim 8, wherein the machine learning model is selected from a logistic regression (LR) model, an artificial neural network (ANN), a Random Forests (RF) model, a support vector machine (SVM), and an ensemble learning (EL) model.

15. A method for classifying a wide complex tachycardia (WCT) pattern of a subject, the method implemented by a computer device comprising at least one processor in communication with at least one memory device, wherein the method comprises:
receiving WCT ECG data indicative of the WCT pattern and baseline ECG data;
transforming the WCT ECG data and baseline ECG data into at least one engineered feature;
executing at least one machine learning model to analyze the at least one engineering feature and to output a classification of the WCT pattern;
based upon the classification of the WCT pattern, determining whether the WCT pattern is indicative of at least one of a ventricular tachycardia (VT) and a supraventricular wide complex tachycardia (SWCT); and
selecting a treatment for the subject based upon the determination.

16. The method of claim 15 further comprising receiving the WCT ECG data from a 12 lead ECG device.

17. The method of claim 15 further comprising determining whether the WCT pattern is indicative of at least one of a ventricular tachycardia, a supraventricular wide complex tachycardia, a probability of a VT, and a probability of an SWCT.

18. The method of claim 15, wherein the at least one engineered feature includes at least one of WCT QRS duration (ms), PMonoTVA (%), QRS-PS for each lead of the ECG data, and any combination thereof.

19. The method of claim 18 further comprising selecting at least one engineering feature for the transformation.

20. The method of claim 18, wherein each QRS-PS for each lead of the ECG data is selected from equiphasic (=)→equiphasic (=), positive (+)→positive (+), negative (−)→negative (−), positive (+)→negative (−), positive (+)→equiphasic (=), negative (−)→positive (+), negative (−)→equiphasic (=), equiphasic (=)→positive (+), or equiphasic (=)→negative (−).

21. The method of claim 15, wherein the machine learning model is selected from a logistic regression (LR) model, an artificial neural network (ANN), a Random Forests (RF) model, a support vector machine (SVM), and an ensemble learning (EL) model.
